SEQUENCE LISTING (16 sequences)

SEQ ID NO: 1 - DNA, 24 bp, Homo sapiens
gtttgtagaccaggaacagttgaa

SEQ ID NO: 2 - DNA, 20 bp, Homo sapiens
cgcatggtgtcctccattat

SEQ ID NO: 3 - DNA, 21 bp, Homo sapiens
cattgtggccaaggagatctg

SEQ ID NO: 4 - DNA, 21 bp, Homo sapiens
cttcggagtttgggtttgctt

SEQ ID NO: 5 - DNA, 22 bp, Homo sapiens
tgtcctttctcagagtggttct

SEQ ID NO: 6 - DNA, 21 bp, Homo sapiens
tgcttccatagggacatcata

SEQ ID NO: 7 - DNA, 22 bp, Homo sapiens
ctctctgctcctcctgttcgac

SEQ ID NO: 8 - DNA, 19 bp, Homo sapiens
tgagcgatgtggctcggct

SEQ ID NO: 9 - DNA, 28 bp, Homo sapiens
cgggcatcatcctagtcttgctgactgt

SEQ ID NO: 10 - DNA, 28 bp, Homo sapiens
atagtggcagtatgtggggggcatcctc

SEQ ID NO: 11 - DNA, 19 bp, Homo sapiens
ggtccctgtcatgcttctg

SEQ ID NO: 12 - DNA, 21 bp, Homo sapiens
tggttgtggaaaaggtagtgg

SEQ ID NO: 13 - DNA, 22 bp, Homo sapiens
caagagctacagaaggatcacc

SEQ ID NO: 14 - DNA, 21 bp, Homo sapiens
cactgattcttgcaaagtccc

SEQ ID NO: 15 - DNA, 21 bp, Homo sapiens
caaggtcatcccagagctgaa

SEQ ID NO: 16 - DNA, 19 bp, Homo sapiens
cagatccacgacggacaca

(19) United States
(12) Patent Application Publication
(10) Pub. No.: US 2024/0034788 A1
(43) Pub. Date: Feb. 1, 2024

(54) ABSCOPAL THERAPY FOR CANCER

(71) Applicant: The Board of Trustees of the Leland Stanford Junior University, Stanford, CA (US)

(72) Inventors: Julien Sage, Menlo Park, CA (US); Edward Graves, Stanford, CA (US); Yoko Nishiga, Redwood City, CA (US)

(21) Appl. No.: 18/020,402

(22) PCT Filed: Aug. 18, 2021
(86) PCT No.: PCT/US2021/046548

§ 371 Date: Feb. 8, 2023

Related U.S. Application Data
(60) Provisional application No. 63/068,172, filed on Aug. 20, 2020.

(51) Int. Cl. C07K 16/28; A61N 5/10; A61P 35/04

(52) U.S. Cl. CPC C07K 16/2803; A61N 5/10; A61P 35/04; C07K 16/2896

(57) ABSTRACT

Methods and compositions are provided for the treatment of cancer with a targeted therapy in combination with radiation. Administration of an effective dose or series of doses of a CD47 blocking agent, i.e. an agent that blocks the interaction between CD47 and SIRPα, is combined with radiation therapy to provide for an abscopal effect. In some embodiments the cancer is a metastatic cancer, or a cancer with a high likelihood of metastasis.

### CROSS REFERENCE TO RELATED APPLICATION

The present application is a 371 and claims the benefit of PCT Application No. PCT/US2021/046548, filed Aug. 18, 20221, which claims priority to U.S. Provisional Patent Application No. 63/068,172 filed Aug. 20, 2020, which application is incorporated herein by reference in its entirety.

### STATEMENT REGARDING FEDERALLY SPONSORED RESEARCH

This invention was made with Government support under contract CA213273 awarded by the National Institutes of Health. The Government has certain rights in the invention.

### INCORPORATION BY REFERENCE OF SEQUENCE LISTING

A Sequence Listing is provided herewith as a Sequence Listing text, “STAN-1772_ST25” created on Feb. 8, 2023, and having a size of 2,626 bytes. The contents of the Sequence Listing text are incorporated herein by reference in their entirety.

### BACKGROUND

Targeted therapies, such as antibodies and specific ligands have proven effective at fighting cancer, especially in cases where conventional therapy fails. Even more encouraging is that antibodies for cancer generally operate in a distinct mechanism from traditional chemotherapy or radiotherapy, so they can often be combined with traditional therapies to generate an additive or synergistic effect.

Radiation therapy is a mainstay of cancer treatment, with more than 50% of all cancer patients receiving radiation during the course of their disease. The primary mode of action of radiation is the direct induction of cancer cell death through acute damage to the DNA. The release of tumor antigens by dead or dying cancer cells further leads to the priming of antigen-specific T cells, thereby activating an adaptive immune response against any remaining cancer cells. In addition, cancer cell debris can stimulate the innate immune system, including recruitment of and increased phagocytosis by macrophages. Based on these observations, a promising strategy to deliver more effective and safer radiation therapy to cancer patients is to combine radiation with immunotherapies such as activation of cytotoxic T cells. However, the development of these combination therapies remains in its infancy and needs to limit the inflammatory protumor effects sometimes observed in irradiated tumors.

CD47 is a valuable target for anticancer therapy due to its function as an inhibitor of macrophage phagocytosis as well as its broad expression on a variety of human neoplasms. By binding to signal-regulatory protein α (SIRPα), a receptor expressed on the surface of macrophages, CD47 is able to transduce inhibitory signals that prevent phagocytosis. Blocking the interaction between CD47 and SIRPα with antibodies not only stimulates macrophages to engulf cancer cells in vitro but also exerts robust anticancer effects in vivo. Other CD47 blocking agents include “next-generation” CD47 antagonists that bind and block human CD47 with extraordinarily high affinity.

By disabling the inhibitory signals transduced by SIRPα, high-affinity SIRPα variants can reduce the threshold for macrophage activation and promote phagocytic response driven by tumor-specific antibodies. The degree to which the anticancer activity of a given therapeutic antibody is enhanced by CD47 blockade likely depends on multiple factors, including the levels of antigen expression on the surface of malignant cells, the isotype of its heavy chain, and the orientation assumed by the antibody upon antigen binding, which affects its ability to engage Fc receptors on immune effectors. High-affinity SIRPα monomers represent therefore a rapid, safe and effective alternative to several other approaches, including drug/toxin conjugation strategies, that have been pursued in this direction.

Identification of effective combinations of targeted therapies remain of high interest. The present invention addresses this need.

### SUMMARY OF THE INVENTION

Methods and compositions are provided for the treatment of cancer with a targeted biologic therapy, in combination with radiation. Administration of an effective dose or series of doses of a CD47 blocking agent, i.e. an agent that blocks the interaction between CD47 and SIRPα, is combined with radiation therapy. In some embodiments the CD47 blocking agent is an antibody that specifically binds to CD47. In some embodiments the cancer is a metastatic cancer, or a cancer with a high likelihood of metastasis. Cancer for treatment with the methods described herein may be an advanced stage, e.g. a Stage II, Stage III or Stage IV cancer.

Radiation therapy uses high-energy rays or particles to kill cancer cells, and can be provided in a palliative or curative dose. Radiation therapy in combination with CD47 blockade surprisingly provides for an abscopal effect, where localized radiation causes a reduction in the number of cancer cells, including without limitation metastatic cancer cells, outside of the field of radiation, due to the activity of macrophages. This combination therapy promotes potent local and abscopal anti-tumor effects. The systemic activation of antitumor macrophages following radiation therapy can be particularly important in cancer patients who suffer from metastatic disease.

In some embodiments the cancer is a lung cancer, including without limitation a Stage II, Stage III or Stage IV lung cancer. In some embodiments, the lung cancer is small cell lung cancer (SCLC). In some embodiments the lung cancer is non-small cell lung cancer (NSCLC). In some embodiments the NSCLC is an adenocarcinoma, a squamous cell carcinoma or a large cell carcinoma.

In some embodiments, the cancer is a colorectal cancer, including without limitation, colorectal adenocarcinoma, carcinoid tumor, gastrointestinal stromal tumor, Turcot Syndrome, Peutz-Jeghers Syndrome, Familial Colorectal Cancer, Juvenile Polyposis Coli, etc. In some embodiments, the cancer is a lymphoma, including without limitation, Hodgkin Lymphoma, Non-Hodgkin Lymphoma, Burkitt Lymphoma, etc.

A CD47 blocking agent for administration to a patient may mask the CD47 protein, e.g. an antibody, polypeptide or small molecule that binds to CD47 or SIRPα and prevents interaction between CD47 and SIRPα. In some embodiments the CD47 blocking agent is an antibody that specifically binds to CD47. In some embodiments an anti-CD47 antibody is a human or humanized antibody, including without limitation an antibody comprising an Fc sequence, e.g. IgG1, IgG2a, IgG2b, IgG3, IgG4 antibody. In some embodiments an anti-CD47 antibody is a humanized IgG4 antibody. In some embodiments an anti-CD47 antibody is magrolimab.

In some embodiments the CD47 blocking agent is an antibody that specifically binds to SIRPα, without activating SIRPα signaling. In some embodiments an anti-SIRPα antibody is a human or humanized antibody, including without limitation an antibody comprising an Fc sequence, e.g. IgG1, IgG2a, IgG2b, IgG3, IgG4 antibody.

In some embodiments the radiation therapy is external beam radiation therapy, including without limitation stereotactic body radiation therapy (SBRT); three-dimensional conformal radiation therapy (3D-CRT); intensity modulated radiation therapy (IMRT), volumetric modulated arc therapy (VMAT). In some embodiments a conventional fractionation scheme of up to 60 or 66 Gy is delivered daily in fractions of from 2 to 2.75 Gray (Gy). In other embodiments the radiation is delivered by hyperfractionation scheme, accelerated fractionation scheme, accelerated hyperfractionation scheme, or hypofractionation scheme.

The CD47 blocking agent can be administered prior to or during the course of radiation. In some embodiments the CD47 blocking agent is delivered concurrently with radiation. In some embodiments, e.g. where radiation is delivered as a course of therapy over multiple days, the CD47 locking agent may be administered on days alternating with the radiation therapy, as desired for the dosage. For example, the CD47 blocking agent may be delivered every other day, every third day, twice a week, once a week, etc., one days that are the same or different as the radiation therapy days.

The therapy may be further combined with additional therapeutic regimens. In some embodiments, one or more additional therapeutic agents can be administered in conjunction with the CD47 blocking agent. Therapeutic agents that find use in the present disclosure, include without limitation, immunotherapy, hormone therapy, chemotherapy, etc. When immunotherapy is used, an immune checkpoint inhibitor may be administered. Immune checkpoint inhibitors that find use in the present disclosure include without limitation, CTLA blocking agents, PD-1/PD-L1 blocking agents, LAG-3 blocking agents, etc. In some embodiments, the PD-1/PD-L1 blocking agent is monoclonal anti-PD-1 antibody clone RMP1-14.

### BRIEF DESCRIPTION OF THE DRAWINGS

FIG. 1. CD47 blockade enhances local tumor inhibition following irradiation of SCLC. (A) Schematic of the analysis of the response of SCLC cells to radiation (RT) in culture. (B) Heat map of relative mRNA levels for Csf-1, Ccl2, and Mcp3 in irradiated KP1 and KP3 mouse SCLC cells and NCI-H82 (H82) human SCLC cells compared non-irradiated control cells. N=2 independent experiments (average values are shown). (C) Flow cytometry analysis of in vitro phagocytosis assays with bone marrow-derived macrophages (BMDMs) and KP1 cells fluorescently labeled with Calcein-AM. N=4 independent experiments shown as the average of technical triplicates. (D) Growth curves of KP1 SCLC allografts in immunodeficient NSG mice with the indicated treatments. (E) Tumor-infiltrating macrophages (CD11b⁺F4/80⁺) identified by flow cytometry from tumors collected in (D). (F) Quantification of tumor-infiltrating macrophages from (E). Data are representative of N=2 independent experiments with n=4-5 tumors per experiment. (G) Growth curves of KP1 SCLC allografts in immunocompetent recipient mice with the indicated treatments. N=1 experiment with n=3-4 tumors. (H) Growth curves of KP1 control and Cd47 knockout SCLC allografts in B6129SF1 immunocompetent syngeneic mice. (I) Quantification of tumor volume 8 days after radiation. N=1 experiment with n=4 tumors. Unpaired t-tests were performed in (C). T-tests following two-way ANOVA were performed in (D) (p<0.0001) and (G) (p<0.0001). T-tests following one-way ANOVA were performed in (F) (right, p=0.001) and (I) (right, p<0.0001). Error bars represent SEM. *p<0.05, **p<0.01, ***p<0.001, ****p<0.0001.

FIG. 2. The combination of radiation therapy and CD47 blockade has an abscopal effect independent of T cells. (A) Mouse KP1 SCLC cells were engrafted into both flanks of B6129SF1 immunocompetent syngeneic mice and only right-side tumors were irradiated. (B) Growth curves of KP1 SCLC allografts as in (A) with the indicated treatments. Note the abscopal effect in the unirradiated tumors. N=1 experiment with n=4-5 mice. (C) Depletion of CD8⁺ T cells following anti-CD8 antibody treatment measured by flow cytometry from the spleen of mice. N=1 experiment with n=5 tumors, error bars represent SEM. (D) Growth curves of KP1 SCLC allografts as in (A) with the indicated treatments. Note the abscopal effect in the unirradiated tumors is not abrogated by depletion of CD8⁺ T cells. N=1 experiment with n=4-5 mice (2 tumors per mouse). See independent experiment in Fig. S5D. (E) Human NCI-H82 and NHJ29 SCLC cells were engrafted into NSG immunocompromised mice and only NHJ29 tumors were irradiated. (F) Growth curves of SCLC xenografts as in (E) with the indicated treatments. Note the abscopal effect in the unirradiated NCI-H82 tumors. N=1 experiment with n=5 mice. Unpaired t-tests were performed in (C). T-tests following two-way ANOVA were performed in (B) (irradiated tumor: p<0.0001, non-irradiated tumor: p=0.0003), (D) (irradiated tumor: p<0.0001, non-irradiated tumor: p<0.0001)) and (F) (irradiated tumor: p<0.0001, non-irradiated tumor: p<0.0001). Error bars represent SEM. *p<0.05, **p<0.01, ***p<0.001, ****p<0.0001.

FIG. 3. The abscopal effect is mediated by macrophages. (A) Mouse KP1 SCLC cells were engrafted into both flanks of B6129SF1 immunocompetent syngeneic mice and only right-side tumors were irradiated. (B) Macrophages were depleted using an anti-CSF-1 antibody, as quantified by flow cytometry (CD11b⁺F4/80⁺ cells) from the tumors of mice in the control group with or without anti-CSF-1 antibody on day 17. N=1 experiment with n=5 mice (2 tumors per mouse). (C) Growth curves of KP1 SCLC allografts as in (A-B) with the indicated treatments. Note the abscopal effect in the non-irradiated tumors is abrogated by CSF-1 blockade and depletion of macrophages. N=1 experiment with n=5 mice (2 tumors per mouse). (D) Histological analysis and quantification of macrophages infiltrating irradiated and control tumors as in the control group in (C) by immunostaining for F4/80. Sections were counterstained with hematoxylin. Each symbol represents one field quantified. Scale bar, 100 μm. Unpaired t-tests were performed in (B). T-tests following two-way ANOVA were performed in (C) (irradiated tumor: p<0.0001, non-irradiated tumor: p<0.0001). T-tests following one-way ANOVA were performed in (E) (irradiated tumors: p<0.0001, non-irradiated tumors: p<0.0001). Error bars represent SEM. *p<0.05, **p<0.01, ***p<0.001, ****p<0.0001.

FIG. 4. The combination of radiation therapy and CD47 blockade has an abscopal effect in multiple cancers. (A) MC38 mouse colon cancer cells were engrafted into both flanks of C57BL/6 immunocompetent syngeneic mice and only right-side tumors were irradiated. (B) Growth curves of MC38 colon allografts as in (A) with the indicated treatments. Note the abscopal effect in the unirradiated tumors. N=1 experiment with n=6-7 mice. (C) Ramos human lymphoma cells were engrafted into both flanks of immunodeficient NSG mice and only right-side tumors were irradiated. (D) Growth curves of Ramos lymphoma xenografts as in (C) with the indicated treatments. Note the abscopal effect in the unirradiated tumors. N=1 experiment with n=6 mice. T-tests following two-way ANOVA were performed in (B) (irradiated tumor: p<0.0001, non-irradiated tumor: p<0.0001) and (D) (irradiated tumor: p<0.0001, non-irradiated tumor: p<0.0001). Error bars represent SEM. **p<0.01, ***p<0.001.

FIG. 5. Radiation induces gene expression changes associated with stress response and inflammation in SCLC cells. (A) SCLC cells were collected 24 hours after radiation (N=1 experiment with 2 controls and 3 irradiated samples) and analyzed by RNA-seq. Genes in red in this MA plot have a p-adjusted value <0.05. (B) Heatmap depicting genes with log2 fold change >2 and <−2 for controls (C) and irradiated samples (RT). (C) GO of upregulated genes. (D) GO of downregulated genes. See Table S1.

FIG. 6. Irradiation of SCLC cells in culture enhances the phagocytosis and the migration ability of macrophages. (A) Cytokine array using conditioned medium of irradiated and control KP1 cells. Conditioned medium was harvested for assay 24 hours after radiation. N=1 experiment (the average of technical triplicates is shown). See Table S2. (B) In vitro phagocytosis assay with mouse bone marrow-derived macrophages (BMDMs) marked by immunofluorescence by F4/80 (red) and beads conjugated with FITC. The supernatant of irradiated (RT) KP1 cells was compared to control cells. DAPI stains the DNA in blue. Scale bar, 100 μm. (C) Quantification of (B), counting FITC-positive macrophages. N=1 experiment with triplicates (D) Flow cytometry analysis of in vitro phagocytosis assay with BMDMs and KP3 cells labeled with Calcein AM. N=1 experiment with 6 technical replicates. (E) Normalized migration ability of irradiated and control BMDMs cultured with conditioned medium of irradiated and control KP1 cells. N=2 independent experiments with 2-3 replicates. Unpaired t-tests were performed in (C) and (D). Error bars represent SEM. *p<0.05, ****p<0.0001.

FIG. 7. CD47 blockade enhances local antitumor effect following radiation in murine SCLC models. (A) Growth curves of KP1 SCLC allografts in NSG mice irradiated with 0, 5, or 10 Gy. N=1 experiment with n=5 tumors for each condition. (B) CD47 expression for KP1 or KP1 Cd47 knockout cells by flow cytometry. (C) In vitro phagocytosis assay performed with mouse bone marrow-derived macrophages (BMDMs) and KP1 control and Cd47 knockout cells. N=1 experiment with triplicates of the primary cultures. (D) Growth curves of KP1 control and Cd47 knockout allografts in NSG mice (left). (E) Quantification of tumor volume 4 days after radiation (right). N=1 experiment with n=3 tumors. Unpaired t-tests were performed in (C). T-tests following two-way ANOVA were performed in (A) (p<0.0001). T-tests following one-way ANOVA were performed in (E, p=0.0006). Error bars represent SEM. *p<0.05, **p<0.01, ***p<0.001.

FIG. 8. CD47 blockade enhances local antitumor effect following radiation in human SCLC tumors in vivo. (A-D) Experiments with NCI-H82 SCLC xenografts in NSG mice. (A) Growth curves with the indicated treatments. (B) Body weight of mice. (C) Representative H&E (hematoxylin and eosin) of NCI-H82 tumor sections. (D) Histological analysis of macrophage infiltration in SCLC xenografts. Specimens were stained for the macrophage marker, F4/80 (left) and the signal was quantified (right). Scale bar, 100 μm. N=1 experiment with n=4 tumors. (E-H) Same as (A-D) for NJH29 SCLC xenografts. (I) Representative sections of the KP1 allograft sections in FIG. 1G with H&E counterstain. Scale bar, 250 μm. N=1 experiment with n=4-5 tumors. T-tests following two-way ANOVA were performed in (A) (p=0.0004) and (E) (p=0.0007). T-tests following one-way ANOVA were performed in (D) (p<0.0001) and (H) (p<0.0001). Error bars represent SEM. *p<0.05, **p<0.01, ***p<0.001, ****p<0.0001.

FIG. 9. The combination of radiation therapy and CD47 blockade has an abscopal effect independent of T cells in mouse models. (A) Mouse KP1 SCLC cells were engrafted into both sides of flanks of B6129SF1 immunocompetent syngeneic mice and only right-side tumors were irradiated using 5 fractions and a total of 20 Gy. (B) Growth curves of KP1 SCLC allografts with the indicated treatments in irradiated and non-irradiated control tumors. Note the abscopal effect in the non-irradiated tumors. N=1 experiment with n=5 mice (2 tumors per mouse). (C) Mouse KP1 SCLC cells were engrafted into both flanks of B6129SF1 immunocompetent syngeneic mice and only right-side tumors were irradiated. CD8⁺ T cells were depleted with anti-CD8 antibody treatment as indicated. (D) Growth curves of KP1 SCLC allografts as in (C) with the indicated treatments in irradiated and non-irradiated control tumors. Note the abscopal effect in the non-irradiated tumors is not abrogated by depletion of CD8⁺ T cells. N=1 experiment with n=5 mice (2 tumors per mouse). Independent experiment shown in FIG. 2D. (E) Histological analysis and quantification of T cell infiltration in irradiated and control tumors as in FIG. 2D by immunostaining for CD3. Sections were counterstained with hematoxylin. Each symbol represents one field quantified. Scale bar, 100 μm. N=1 experiment with n=4-5 mice (2 tumors per mouse). T-tests following two-way ANOVA were performed in (B) (irradiated tumors: p<0.0001, non-irradiated tumors: p<0.0001), (D) (irradiated tumors: p<0.0001, non-irradiated tumors: p<0.0001). T-tests following one-way ANOVA were performed in (C) (irradiated tumors: p=0.0054, non-irradiated tumors: p=0.30). Error bars represent SEM. *p<0.05, **p<0.01, ***p<0.001, ****p<0.0001.

FIG. 10. The combination of radiation therapy and CD47 blockade has an abscopal effect independent of T cells in mouse models. (A) Human NJH29 SCLC cells were engrafted into both sides of NSG mice and only right-side tumors were irradiated. Growth curves of NJH29 xenografts with the indicated treatments. Note the abscopal effect in the unirradiated tumors. N=1 experiment with n=5 mice (2 tumors per mouse). (C-D) Same as (A-B) with NCI-H82 SCLC cells. N=1 experiment with n=4-5 mice (2 tumors per mouse). (E) Histological analysis and quantification of macrophages infiltrating irradiated and control tumors as in (D) by immunostaining for F4/80. Sections were counterstained with hematoxylin. Each symbol represents one field quantified. Scale bar, 100 μm. stats T-tests following two-way ANOVA were performed in (B) (irradiated tumors: p=0.0015, non-irradiated tumors: p=0.0055) and (D) (irradiated tumors: p<0.0001, non-irradiated tumors: p<0.0001). T-tests following one-way ANOVA were performed in (E) (irradiated tumors: p<0.0001, non-irradiated tumors: p<0.0001). Error bars represent SEM. *p<0.05, **p<0.01, ***p<0.001, ****p<0.0001.

FIG. 11. Abscopal effect of combination of radiation and CD47 blockade is mediated by macrophages. (A) Macrophages were depleted with anti-CSF-1 antibody treatment as indicated. (B) Mouse KP1 SCLC cells were engrafted into both sides of flanks of NSG mice and only right-side tumors were irradiated. (C) Growth curves of KP1 SCLC allografts as in (A-B) with the indicated treatments. Note the abscopal effect in the non-irradiated tumors is abrogated by CSF-1 blockade and depletion of macrophages. N=1 experiment with n=5 tumors. T-tests following two-way ANOVA were performed in (C) (irradiated tumors: p<0.0001, non-irradiated tumors: p<0.0001). Error bars represent SEM. *p<0.05, **p<0.01, ***p<0.001, ****p<0.0001.

FIG. 12. PD-1 blockade enhances the abscopal effect of combination of radiation therapy and CD47 blockade in mouse colon tumors. (A) MC38 mouse colon cancer cells were engrafted into both flanks of C57BL/6 immunocompetent syngeneic mice and only right-side tumors were irradiated. (B) Growth curves of MC38 colon allografts as in (A) with the indicated treatments. Note the abscopal effect in the unirradiated tumors is enhanced by PD-1 blockade. N=1 experiment with n=7-8 mice. T-tests following two-way ANOVA were performed in (B) (irradiated tumor: p=0.78, non-irradiated tumor: p<0.0001). Error bars represent SEM. ****p<0.0001. Anti-PD-1 antibody (clone RMP1-14, BioXCell, BE0146).

### DETAILED DESCRIPTION OF THE EMBODIMENTS

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Methods recited herein may be carried out in any order of the recited events which is logically possible, as well as the recited order of events.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

Synergistic combination. Synergistic combinations may provide for a therapeutic effect that is comparable to the effectiveness of a monotherapy, i.e. the individual components of the combination, while reducing adverse side effects, e.g. damage to non-targeted tissues, immune status, and other clinical indicia. Alternatively synergistic combinations may provide for an improved effectiveness when compared to the effectiveness of a monotherapy, i.e. the individual components of the combination, which effect may be measured by decreased metastasis, total tumor cell number; length of time to relapse; and other indicia of patient health.

Combination Therapy: As used herein, the term “combination therapy” refers to those situations in which a subject is simultaneously exposed to two or more therapeutic regimens (e.g., two or more therapeutic agents). In some embodiments, two or more agents may be administered simultaneously; in some embodiments, such agents may be administered sequentially; in some embodiments, such agents are administered in overlapping dosing regimens.

Dosage Form: As used herein, the term “dosage form” refers to a physically discrete unit of an active agent (e.g., a therapeutic or diagnostic agent) for administration to a subject. Each unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Dosing Regimen: As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount In some embodiments, a dosing regimen is correlated with a desired or beneficial outcome when administered across a relevant population (i.e., is a therapeutic dosing regimen).

Abscopal effect: As used herein, the term “abscopal effect” refers to a physiological process whereby targeted radiation of a primary tumor induces an anti-tumor response at a distant site that is not in the field of radiation. The effect can be particularly important for the treatment of metastatic cancer.

Anti-CD47 agent. CD47 is a broadly expressed transmembrane glycoprotein with a single Ig-like domain and five membrane spanning regions, which functions as a cellular ligand for SIRPα with binding mediated through the NH2-terminal V-like domain of SIRPα. SIRPα is expressed primarily on myeloid cells, including macrophages, granulocytes, myeloid dendritic cells (DCs), mast cells, and their precursors, including hematopoietic stem cells. Structural determinants on SIRPα that mediate CD47 binding are discussed by Lee et al. (2007) J. Immunol. 179:7741-7750; Hatherley et al. (2008) Mol Cell. 31(2):266-77; Hatherley et al. (2007) J. B. C. 282:14567-75; and the role of SIRPα cis dimerization in CD47 binding is discussed by Lee et al. (2010) J. B. C. 285:37953-63. In keeping with the role of CD47 to inhibit phagocytosis of normal cells, there is evidence that it is transiently upregulated on hematopoietic stem cells (HSCs) and progenitors just prior to and during their migratory phase, and that the level of CD47 on these cells determines the probability that they are engulfed in vivo.

As used herein, the term “anti-CD47 agent” or “agent that provides for CD47 blockade” or “CD47 blocking agent” refers to any agent that reduces the binding of CD47 (e.g., on a target cell) to SIRPα (e.g., on a phagocytic cell). Non-limiting examples of suitable anti-CD47 reagents include without limitation high affinity SIRPα polypeptides, anti-SIRPα antibodies, soluble CD47 polypeptides, and anti-CD47 antibodies or antibody fragments. In some embodiments, a suitable anti-CD47 agent (e.g. an anti-CD47 antibody, a SIRPα reagent, etc.) specifically binds CD47 to reduce the binding of CD47 to SIRPα.

In some embodiments, a suitable anti-CD47 agent (e.g., an anti-SIRPα antibody, a soluble CD47 polypeptide, etc.) specifically binds SIRPα to reduce the binding of CD47 to SIRPα. A suitable anti-CD47 agent that binds SIRPα does not activate SIRPα (e.g., in the SIRPα-expressing phagocytic cell). The efficacy of a suitable anti-CD47 agent can be assessed by assaying the agent. In an exemplary assay, target cells are incubated in the presence or absence of the candidate agent and in the presence of an effector cell, e.g. a macrophage or other phagocytic cell. An agent for use in the methods of the invention will up-regulate phagocytosis by at least 5% (e.g., at least 10%, at least 20%, at least 30%, at least 40%, at least 50%, at least 60%, at least 70%, at least 80%, at least 90%, at least 100%, at least 120%, at least 140%, at least 160%, at least 180%, at least 200%, at least 500%, at least 1000%) compared to phagocytosis in the absence of the agent. Similarly, an in vitro assay for levels of tyrosine phosphorylation of SIRPα will show a decrease in phosphorylation by at least 5% (e.g., at least 10%, at least 15%, at least 20%, at least 30%, at least 40%, at least 50%, at least 60%, at least 70%, at least 80%, at least 90%, or 100%) compared to phosphorylation observed in absence of the candidate agent.

In some embodiments, the anti-CD47 agent does not activate CD47 upon binding. When CD47 is activated, a process akin to apoptosis (i.e., programmed cell death) may occur (Manna and Frazier, Cancer Research, 64, 1026-1036, Feb. 1, 2004). Thus, in some embodiments, the anti-CD47 agent does not directly induce cell death of a CD47-expressing cell.

In some embodiments a primer agent is administered prior to administering a therapeutically effective dose of an anti-CD47 agent to the individual. Suitable primer agents include an erythropoiesis-stimulating agent (ESA), and/or a sub-therapeutic, priming dose of an anti-CD47 agent. Following administration of the priming agent, and allowing a period of time effective for an increase in reticulocyte production, a therapeutic dose of an anti-CD47 agent is administered. Administration may be made in accordance with the methods described in co-pending patent application U.S. Ser. No. 14/769,069, herein specifically incorporated by reference.

SIRPα reagent. A SIRPα reagent comprises the portion of SIRPα that is sufficient to bind CD47 at a recognizable affinity, which normally lies between the signal sequence and the transmembrane domain, or a fragment thereof that retains the binding activity. A suitable SIRPα reagent reduces (e.g., blocks, prevents, etc.) the interaction between the native proteins SIRPα and CD47. The SIRPα reagent will usually comprise at least the d1 domain of SIRPα.

In some embodiments, a subject anti-CD47 agent is a “high affinity SIRPα reagent”, which includes SIRPα-derived polypeptides and analogs thereof (e.g., CV1-hIgG4, and CV1 monomer). High affinity SIRPα reagents are described in international application PCT/US13/21937, which is hereby specifically incorporated by reference. High affinity SIRPα reagents are variants of the native SIRPα protein. The amino acid changes that provide for increased affinity are localized in the d1 domain, and thus high affinity SIRPα reagents comprise a d1 domain of human SIRPα, with at least one amino acid change relative to the wild-type sequence within the d1 domain. Such a high affinity SIRPα reagent optionally comprises additional amino acid sequences, for example antibody Fc sequences; portions of the wild-type human SIRPα protein other than the d1 domain, including without limitation residues 150 to 374 of the native protein or fragments thereof, usually fragments contiguous with the d1 domain; and the like. High affinity SIRPα reagents may be monomeric or multimeric, i.e. dimer, trimer, tetramer, etc. In some embodiments, a high affinity SIRPα reagent is soluble, where the polypeptide lacks the SIRPα transmembrane domain and comprises at least one amino acid change relative to the wild-type SIRPα sequence, and wherein the amino acid change increases the affinity of the SIRPα polypeptide binding to CD47, for example by decreasing the off-rate by at least 10-fold, at least 20-fold, at least 50-fold, at least 100-fold, at least 500-fold, or more.

Optionally the SIRPα reagent is a fusion protein, e.g., fused in frame with a second polypeptide. In some embodiments, the second polypeptide is capable of increasing the size of the fusion protein, e.g., so that the fusion protein will not be cleared from the circulation rapidly. In some embodiments, the second polypeptide is part or whole of an immunoglobulin Fc region. The Fc region aids in phagocytosis by providing an “eat me” signal, which enhances the block of the “don't eat me” signal provided by the high affinity SIRPα reagent. In other embodiments, the second polypeptide is any suitable polypeptide that is substantially similar to Fc, e.g., providing increased size, multimerization domains, and/or additional binding or interaction with Ig molecules.

Anti-CD47 antibodies. In some embodiments, a subject anti-CD47 agent is an antibody that specifically binds CD47 (i.e., an anti-CD47 antibody) and reduces the interaction between CD47 on one cell (e.g., an infected cell) and SIRPα on another cell (e.g., a phagocytic cell). In some embodiments, a suitable anti-CD47 antibody does not activate CD47 upon binding. Some anti-CD47 antibodies do not reduce the binding of CD47 to SIRPα (and are therefore not considered to be an “anti-CD47 agent” herein) and such an antibody can be referred to as a “non-blocking anti-CD47 antibody.” A suitable anti-CD47 antibody that is an “anti-CD47 agent” can be referred to as a “CD47-blocking antibody”. Non-limiting examples of suitable antibodies include clones B6H12, 5F9, 8B6, and C3 (for example as described in International Patent Publication WO 2011/143624, herein specifically incorporated by reference). Suitable anti-CD47 antibodies include fully human, humanized or chimeric versions of such antibodies. Humanized antibodies (e.g., hu5F9-G4, magrolimab) are especially useful for in vivo applications in humans due to their low antigenicity. Similarly caninized, felinized, etc. antibodies are especially useful for applications in dogs, cats, and other species respectively. Antibodies of interest include humanized antibodies, or caninized, felinized, equinized, bovinized, porcinized, etc., antibodies, and variants thereof.

In some embodiments an anti-CD47 antibody comprises a human IgG Fc region, e.g. an IgG1, IgG2a, IgG2b, IgG3, IgG4 constant region. In a preferred embodiment the IgG Fc region is an IgG4 constant region. The IgG4 hinge may be stabilized by the amino acid substitution S241P (see Angal et al. (1993) Mol. Immunol. 30(1):105-108, herein specifically incorporated by reference).

Anti-SIRPα antibodies. In some embodiments, a subject anti-CD47 agent is an antibody that specifically binds SIRPα (i.e., an anti-SIRPα antibody) and reduces the interaction between CD47 on one cell (e.g., an infected cell) and SIRPα on another cell (e.g., a phagocytic cell). Suitable anti-SIRPα antibodies can bind SIRPα without activating or stimulating signaling through SIRPα because activation of SIRPα would inhibit phagocytosis. Instead, suitable anti-SIRPα antibodies facilitate the preferential phagocytosis of inflicted cells over normal cells. Those cells that express higher levels of CD47 (e.g., infected cells) relative to other cells (non-infected cells) will be preferentially phagocytosed. Thus, a suitable anti-SIRPα antibody specifically binds SIRPα (without activating/stimulating enough of a signaling response to inhibit phagocytosis) and blocks an interaction between SIRPα and CD47. Suitable anti-SIRPα antibodies include fully human, humanized or chimeric versions of such antibodies. Humanized antibodies are especially useful for in vivo applications in humans due to their low antigenicity. Similarly caninized, felinized, etc. antibodies are especially useful for applications in dogs, cats, and other species respectively. Antibodies of interest include humanized antibodies, or caninized, felinized, equinized, bovinized, porcinized, etc., antibodies, and variants thereof.

Soluble CD47 polypeptides. In some embodiments, a subject anti-CD47 agent is a soluble CD47 polypeptide that specifically binds SIRPα and reduces the interaction between CD47 on one cell (e.g., an infected cell) and SIRPα on another cell (e.g., a phagocytic cell). A suitable soluble CD47 polypeptide can bind SIRPα without activating or stimulating signaling through SIRPα because activation of SIRPα would inhibit phagocytosis. Instead, suitable soluble CD47 polypeptides facilitate the preferential phagocytosis of infected cells over non-infected cells. Those cells that express higher levels of CD47 (e.g., infected cells) relative to normal, non-target cells (normal cells) will be preferentially phagocytosed. Thus, a suitable soluble CD47 polypeptide specifically binds SIRPα without activating/stimulating enough of a signaling response to inhibit phagocytosis.

In some cases, a suitable soluble CD47 polypeptide can be a fusion protein (for example as structurally described in US Patent Publication US20100239579, herein specifically incorporated by reference). However, only fusion proteins that do not activate/stimulate SIRPα are suitable for the methods provided herein. Suitable soluble CD47 polypeptides also include any peptide or peptide fragment comprising variant or naturally existing CD47 sequences (e.g., extracellular domain sequences or extracellular domain variants) that can specifically bind SIRPα and inhibit the interaction between CD47 and SIRPα without stimulating enough SIRPα activity to inhibit phagocytosis.

In certain embodiments, soluble CD47 polypeptide comprises the extracellular domain of CD47, including the signal peptide, such that the extracellular portion of CD47 is typically 142 amino acids in length. The soluble CD47 polypeptides described herein also include CD47 extracellular domain variants that comprise an amino acid sequence at least 65%-75%, 75%-80%, 80-85%, 85%-90%, or 95%-99% (or any percent identity not specifically enumerated between 65% to 100%), which variants retain the capability to bind to SIRPα without stimulating SIRPα signaling.

In certain embodiments, the signal peptide amino acid sequence may be substituted with a signal peptide amino acid sequence that is derived from another polypeptide (e.g., for example, an immunoglobulin or CTLA4). For example, unlike full-length CD47, which is a cell surface polypeptide that traverses the outer cell membrane, the soluble CD47 polypeptides are secreted; accordingly, a polynucleotide encoding a soluble CD47 polypeptide may include a nucleotide sequence encoding a signal peptide that is associated with a polypeptide that is normally secreted from a cell.

In other embodiments, the soluble CD47 polypeptide comprises an extracellular domain of CD47 that lacks the signal peptide. As described herein, signal peptides are not exposed on the cell surface of a secreted or transmembrane protein because either the signal peptide is cleaved during translocation of the protein or the signal peptide remains anchored in the outer cell membrane (such a peptide is also called a signal anchor). The signal peptide sequence of CD47 is believed to be cleaved from the precursor CD47 polypeptide in vivo.

In other embodiments, a soluble CD47 polypeptide comprises a CD47 extracellular domain variant. Such a soluble CD47 polypeptide retains the capability to bind to SIRPα without stimulating SIRPα signaling. The CD47 extracellular domain variant may have an amino acid sequence that is at least 65%-75%, 75%-80%, 80-85%, 85%-90%, or 95%-99% identical (which includes any percent identity between any one of the described ranges) to the native CD47 sequence.

The term “immune checkpoint inhibitor” refers to a molecule, compound, or composition that binds to an immune checkpoint protein and blocks its activity and/or inhibits the function of the immune regulatory cell expressing the immune checkpoint protein that it binds (e.g., Treg cells, tumor-associated macrophages, etc.). Immune checkpoint proteins may include, but are not limited to, CTLA4 (Cytotoxic T-Lymphocyte-Associated protein 4, CD152), PD1 (also known as PD-1; Programmed Death 1 receptor), PD-L1, PD-L2, LAG-3 (Lymphocyte Activation Gene-3), OX40, A2AR (Adenosine A2A receptor), B7-H3 (CD276), B7-H4 (VTCN1), BTLA (B and T Lymphocyte Attenuator, CD272), IDO (Indoleamine 2,3-dioxygenase), KIR (Killer-cell Immunoglobulin-like Receptor), TIM 3 (T-cell Immunoglobulin domain and Mucin domain 3), VISTA (V-domain Ig suppressor of T cell activation), and IL-2R (interleukin-2 receptor).

Immune checkpoint inhibitors are well known in the art and are commercially or clinically available. These include but are not limited to antibodies that inhibit immune checkpoint proteins. Illustrative examples of checkpoint inhibitors, referenced by their target immune checkpoint protein, are provided as follows. Immune checkpoint inhibitors comprising a CTLA-4 inhibitor include, but are not limited to, tremelimumab, and ipilimumab (marketed as Yervoy).

Immune checkpoint inhibitors comprising a PD-1 inhibitor include, but are not limited to, nivolumab (Opdivo), pidilizumab (CureTech), AMP-514 (Medlmmune), pembrolizumab (Keytruda), AUNP 12 (peptide, Aurigene and Pierre), Cemiplimab (Libtayo). Immune checkpoint inhibitors comprising a PD-L1 inhibitor include, but are not limited to, BMS-936559/MDX-1105 (Bristol-Myers Squibb), MPDL3280A (Genentech), MEDI4736 (Medlmmune), MSB0010718C (EMD Sereno), Atezolizumab (Tecentriq), Avelumab (Bavencio), Durvalumab (Imfinzi).

Immune checkpoint inhibitors comprising a B7-H3 inhibitor include, but are not limited to, MGA271 (Macrogenics). Immune checkpoint inhibitors comprising an LAGS inhibitor include, but are not limited to, IMP321 (Immuntep), BMS-986016 (Bristol-Myers Squibb). Immune checkpoint inhibitors comprising a KIR inhibitor include, but are not limited to, IPH2101 (Iirilumab, Bristol-Myers Squibb). Immune checkpoint inhibitors comprising an OX40 inhibitor include, but are not limited to MEDI-6469 (Medlmmune). An immune checkpoint inhibitor targeting IL-2R, for preferentially depleting Treg cells (e.g., FoxP-3+ CD4+ cells), comprises IL-2-toxin fusion proteins, which include, but are not limited to, denileukin diftitox (Ontak; Eisai).

As used herein, “antibody” includes reference to an immunoglobulin molecule immunologically reactive with a particular antigen, and includes both polyclonal and monoclonal antibodies. The term also includes genetically engineered forms such as chimeric antibodies (e.g., humanized murine antibodies) and heteroconjugate antibodies. The term “antibody” also includes antigen binding forms of antibodies, including fragments with antigen-binding capability (e.g., Fab′, F(ab′)₂, Fab, Fv and rIgG. The term also refers to recombinant single chain Fv fragments (scFv). The term antibody also includes bivalent or bispecific molecules, diabodies, triabodies, and tetrabodies.

Selection of antibodies may be based on a variety of criteria, including selectivity, affinity, cytotoxicity, etc. The phrase “specifically (or selectively) binds” to an antibody or “specifically (or selectively) immunoreactive with,” when referring to a protein or peptide, refers to a binding reaction that is determinative of the presence of the protein, in a heterogeneous population of proteins and other biologics. Thus, under designated immunoassay conditions, the specified antibodies bind to a particular protein sequences at least two times the background and more typically more than 10 to 100 times background. In general, antibodies of the present invention bind antigens on the surface of target cells in the presence of effector cells (such as natural killer cells or macrophages). Fc receptors on effector cells recognize bound antibodies.

An antibody immunologically reactive with a particular antigen can be generated by recombinant methods such as selection of libraries of recombinant antibodies in phage or similar vectors, or by immunizing an animal with the antigen or with DNA encoding the antigen. Methods of preparing polyclonal antibodies are known to the skilled artisan. The antibodies may, alternatively, be monoclonal antibodies. Monoclonal antibodies may be prepared using hybridoma methods. In a hybridoma method, an appropriate host animal is typically immunized with an immunizing agent to elicit lymphocytes that produce or are capable of producing antibodies that will specifically bind to the immunizing agent. Alternatively, the lymphocytes may be immunized in vitro. The lymphocytes are then fused with an immortalized cell line using a suitable fusing agent, such as polyethylene glycol, to form a hybridoma cell.

Human antibodies can be produced using various techniques known in the art, including phage display libraries. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. Antibodies also exist as a number of well-characterized fragments produced by digestion

with various peptidases. Thus pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into a Fab′ monomer. The Fab′ monomer is essentially Fab with part of the hinge region. While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries.

A “humanized antibody” is an immunoglobulin molecule which contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin.

Antibodies of interest may be tested for their ability to induce ADCC (antibody-dependent cellular cytotoxicity) or ADCP (antibody dependent cellular phagocytosis). Antibody-associated ADCC activity can be monitored and quantified through detection of either the release of label or lactate dehydrogenase from the lysed cells, or detection of reduced target cell viability (e.g. annexin assay). Assays for apoptosis may be performed by terminal deoxynucleotidyl transferase-mediated digoxigenin-11-dUTP nick end labeling (TUNEL) assay (Lazebnik et al., Nature: 371, 346 (1994). Cytotoxicity may also be detected directly by detection kits known in the art, such as Cytotoxicity Detection Kit from Roche Applied Science (Indianapolis, Ind.).

The terms “subject,” “individual,” and “patient” are used interchangeably herein to refer to a mammal being assessed for treatment and/or being treated. In an embodiment, the mammal is a human. The terms “subject,” “individual,” and “patient” encompass, without limitation, individuals having cancer, e.g. metastatic cancer. Subjects may be human, but also include other mammals, particularly those mammals useful as laboratory models for human disease, e.g. mouse, rat, etc.

The terms “cancer,” “neoplasm,” and “tumor” are used interchangeably herein to refer to cells which exhibit autonomous, unregulated growth, such that they exhibit an aberrant growth phenotype characterized by a significant loss of control over cell proliferation. Cells of interest for detection, analysis, or treatment in the present application include precancerous (e.g., benign), malignant, pre-metastatic, metastatic, and non-metastatic cells. Cancers of virtually every tissue are known. The phrase “cancer burden” refers to the quantum of cancer cells or cancer volume in a subject. Reducing cancer burden accordingly refers to reducing the number of cancer cells or the cancer volume in a subject. The term “cancer cell” as used herein refers to any cell that is a cancer cell or is derived from a cancer cell e.g. clone of a cancer cell.

The types of cancer that can be treated using the subject methods of the present invention include but are not limited to adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's lymphoma, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, non-melanoma skin cancers, stomach cancer, testicular cancer, thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, or Waldenstrom's macroglobulinemia.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

As used herein, the terms “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs; therefore tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable number of cancerous cells in an organ or body part which is not directly connected to the organ of the original cancerous tumor. Metastasis can also be defined as several steps of a process, such as the departure of cancer cells from an original tumor site, and migration and/or invasion of cancer cells to other parts of the body.

The term “sample” with respect to a patient encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as cancer cells. The definition also includes sample that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's cancer cell, e.g., a sample comprising polynucleotides and/or polypeptides that is obtained from a patient's cancer cell (e.g., a cell lysate or other cell extract comprising polynucleotides and/or polypeptides); and a sample comprising cancer cells from a patient. A biological sample comprising a cancer cell from a patient can also include non-cancerous cells.

The term “diagnosis” is used herein to refer to the identification of a molecular or pathological state, disease or condition.

The term “prognosis” is used herein to refer to the prediction of the likelihood of cancer-attributable death or progression, including recurrence, metastatic spread, and drug resistance, of a neoplastic disease, such as ovarian cancer. The term “prediction” is used herein to refer to the act of foretelling or estimating, based on observation, experience, or scientific reasoning. In one example, a physician may predict the likelihood that a patient will survive, following surgical removal of a primary tumor and/or chemotherapy for a certain period of time without cancer recurrence.

As used herein, the terms “treatment,” “treating,” and the like, refer to administering an agent, or carrying out a procedure, for the purposes of obtaining an effect. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of a tumor in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease.

Treating may refer to any indicia of success in the treatment or amelioration or prevention of an cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with cancer or other diseases. The term “therapeutic effect” refers to the reduction, elimination, or prevention of the disease, symptoms of the disease, or side effects of the disease in the subject.

“In combination with”, “combination therapy” and “combination products” refer, in certain embodiments, to the concurrent administration to a patient of a first therapeutic and the compounds as used herein. When administered in combination, each component can be administered at the same time or sequentially in any order at different points in time. Thus, each component can be administered separately but sufficiently closely in time so as to provide the desired therapeutic effect.

“Concomitant administration” , e.g. or radiation therapy an administration of a CD47 blocking agent, means administration at such time that both will have a therapeutic effect. Such concomitant administration may involve concurrent (i.e. at the same time), prior, or subsequent administration of the radiation or CD47 blocking agent. A person of ordinary skill in the art would have no difficulty determining the appropriate timing, sequence and dosages of administration for particular drugs and compositions of the present invention.

As used herein, endpoints for treatment will be given a meaning as known in the art and as used by the Food and Drug Administration.

Overall survival is defined as the time from randomization until death from any cause, and is measured in the intent-to-treat population. Survival is considered the most reliable cancer endpoint, and when studies can be conducted to adequately assess survival, it is usually the preferred endpoint. This endpoint is precise and easy to measure, documented by the date of death. Bias is not a factor in endpoint measurement. Survival improvement should be analyzed as a risk-benefit analysis to assess clinical benefit. Overall survival can be evaluated in randomized controlled studies. Demonstration of a statistically significant improvement in overall survival can be considered to be clinically significant if the toxicity profile is acceptable, and has often supported new drug approval. A benefit of the methods of the invention can include increased overall survival of patients.

Endpoints that are based on tumor assessments include DFS, ORR, TTP, PFS, and time-to-treatment failure (TTF). The collection and analysis of data on these time-dependent endpoints are based on indirect assessments, calculations, and estimates (e.g., tumor measurements). Disease-Free Survival (DFS) is defined as the time from randomization until recurrence of tumor or death from any cause. The most frequent use of this endpoint is in the adjuvant setting after definitive surgery or radiotherapy. DFS also can be an important endpoint when a large percentage of patients achieve complete responses with chemotherapy.

Objective Response Rate . ORR is defined as the proportion of patients with tumor size reduction of a predefined amount and for a minimum time period. Response duration usually is measured from the time of initial response until documented tumor progression. Generally, the FDA has defined ORR as the sum of partial responses plus complete responses. When defined in this manner, ORR is a direct measure of drug antitumor activity, which can be evaluated in a single-arm study.

Time to Progression and Progression-Free Survival. TTP and PFS have served as primary endpoints for drug approval. TTP is defined as the time from randomization until objective tumor progression; TTP does not include deaths. PFS is defined as the time from randomization until objective tumor progression or death. The precise definition of tumor progression is important and should be carefully detailed in the protocol.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

“Pharmaceutically acceptable salts and esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g., ethanolamine, diethanolamine, triethanolamine, tromethamine, N methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g., C₁₋₆ alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention may be present in more than one stereoisomeric form, and the naming of such compounds is intended to include all single stereoisomers and all mixtures (whether racemic or otherwise) of such stereoisomers.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” means the amount that, when administered to a subject for treating a disease, is sufficient to effect treatment for that disease.

### Lung Cancer

Lung carcinoma is the leading cause of cancer-related death worldwide. About 85% of cases are related to cigarette smoking. Symptoms can include cough, chest discomfort or pain, weight loss, and, less commonly, hemoptysis; however, many patients present with metastatic disease without any clinical symptoms. The diagnosis is typically made by chest x-ray or CT and confirmed by biopsy. Depending on the stage of the disease, treatment includes surgery, chemotherapy, radiation therapy, or a combination. For the past several decades, the prognosis for a lung cancer patient has been poor, particularly for patients with stage IV (metastatic) disease.

Respiratory epithelial cells require prolonged exposure to cancer-promoting agents and accumulation of multiple genetic mutations before becoming neoplastic (an effect called field carcinogenesis). In some patients with lung cancer, secondary or additional mutations in genes that stimulate cell growth (K-ras, MYC), cause abnormalities in growth factor receptor signaling (EGFR, HER2/neu), and inhibit apoptosis contribute to proliferation of abnormal cells. In addition, mutations that inhibit tumor-suppressor genes (p53, APC) can lead to cancer. Other mutations that may be responsible include the EML-4-ALK translocation and mutations in ROS-1, BRAF, and PI3KCA. Genes such as these that are primarily responsible for lung cancer are called driver mutations. Although driver mutations can cause or contribute to lung cancer among smokers, these mutations are particularly likely to be a cause of lung cancer among nonsmokers.

Chest x-ray is often the initial imaging test. It may show clearly defined abnormalities, such as a single mass or multifocal masses or a solitary pulmonary nodule, an enlarged hilum, widened mediastinum, tracheobronchial narrowing, atelectasis, non-resolving parenchymal infiltrates, cavitary lesions, or unexplained pleural thickening or effusion. These findings are suggestive but not diagnostic of lung cancer and require follow-up with CT scans or combined PET-CT scans and cytopathologic confirmation.

CT shows many characteristic anatomic patterns and appearances that may strongly suggest the diagnosis. CT also can guide core needle biopsy of accessible lesions and is useful for staging. If a lesion found on a plain x-ray is highly likely to be lung cancer, PET-CT may be done. This study combines anatomic imaging from CT with functional imaging from PET. The PET images can help differentiate inflammatory and malignant processes.

Lung cancer is classified into 2 major categories: Small cell lung cancer (SCLC), about 15% of cases and Non-small cell lung cancer (NSCLC), about 85% of cases. SCLC is highly aggressive and almost always occurs in smokers. It is rapidly growing, and roughly 80% of patients have metastatic disease at the time of diagnosis. The clinical behavior of NSCLC is more variable and depends on histologic type, but about 40% of patients will have metastatic disease outside of the chest at the time of diagnosis. The histologic type may be large cell, adenocarcinoma, squamous cell carcinomas Oncogenic driver mutations have been identified primarily in adenocarcinoma, and attempts are being made to identify similar mutations in squamous cell carcinoma (eg, FGFR1, DDR2, and PI3K).

SCLC has 2 stages: limited and extensive. Limited-stage SCLC disease is cancer confined to one hemithorax (including ipsilateral lymph nodes) that can be encompassed within one tolerable radiation therapy port, unless there is a pleural or pericardial effusion. Extensive-stage disease is cancer outside a single hemithorax or the presence of malignant cells detected in pleural or pericardial effusions. Less than one third of patients with SCLC will present with limited-stage disease; the remainder of patients often have extensive distant metastases. The overall prognosis for SCLC is poor. The median survival time for limited-stage SCLC is 20 mo, with a 5-yr survival rate of 20%. Patients with extensive-stage SCLC do especially poorly, with a survival rate of <1%.

NSCLC has 4 stages, I through IV (using the TNM system). TNM staging is based on tumor size, tumor and lymph node location, and the presence or absence of distant metastases. The 5-yr survival rate of patients with NSCLC varies by stage, from 60 to 70% for patients with stage I disease to <1% for patients with stage IV disease. NSCLC staging is described below.

TABLE 1
Staging of NSCLC.

| | |
|---|---|
| Category | Description |

Primary Tumor (T)

| | |
|---|---|
| Tis | Carcinoma in situ |
| T1 | Tumor ≤3 cm without invasion more proximal |
| | than the lobar bronchus |
| T1mi | Minimally invasive adenocarcinoma |
| T1a | Tumor ≤1 cm |
| T1b | Tumor >1 cm but ≤2 cm |
| T1c | Tumor >2 cm but ≤3 cm |
| T2 | Tumor >3 cm but ≤5 cm or with any of the following: |
| | Involves the main bronchus ≥2 cm distal to carina |
| | Invades the visceral pleura |
| | Associated with atelectasis or obstructive pneumonia that |
| | extends to the hilar region but, involving part of the |
| | lung or the entire lung |
| T2a | Tumor >3 but ≤4 cm |
| T2b | Tumor >4 but ≤5 cm |
| T3 | Tumor >5 cm but ≤7 cm or with any of the following: |
| | Invades the chest wall (including superior sulcus tumors), |
| | phrenic nerve, parietal pericardium |
| | Separate tumor nodules in the same lobe |
| T4 | Tumor >7 cm or with either of the following: |
| | Invades the diaphragm, mediastinum, heart, great vessels, |
| | trachea, recurrent laryngeal nerve, esophagus, vertebral |
| | body, or carina |
| | ≥1 Satellite tumors in a different ipsilateral lobe |

Regional Lymph nodes (N)

| | |
|---|---|
| N0 | No regional lymph node metastasis |
| N1 | Metastasis to ipsilateral peribronchial or ipsilateral |
| | hilar lymph node or both and to intrapulmonary nodes, |
| | including that by direct extension of the primary tumor |
| N2 | Metastasis to ipsilateral mediastinal or subcarinal |
| | lymph node or both |
| N3 | Metastasis to contralateral mediastinal, contralateral |
| | hilar, ipsilateral or contralateral scalene, or |
| | supraclavicular lymph node or a combination |

Distant metastasis (M)

| | |
|---|---|
| M0 | No distant metastasis |
| M1 | Distant metastasis |
| 67 | Tumor with any of the following: |
| | ≥1 Tumor nodules in the contralateral lung |
| | Pleural or pericardial nodules |
| | Malignant pleural or pericardial effusion |
| M1b | Single extrathoracic metastasis in a single organ |
| M1c | Multiple extrathoracic metastases in one or several organs |

Staging groupings

| | |
|---|---|
| Stage 0 | Tis N0 M0 |
| Stage IA1 | T1mi-T1a N0 M0 |
| Stage IA2 | T1b N0 M0 |
| Stage IA3 | T1c N0 M0 |
| Stage IB | T2a N0 M0 |
| Stage IIA | T2a N0 M0 |
| Stage IIB | T1a-T2b N1 M0 or T3 N0 M0 |
| Stage IIIA | T1a-T2b N2 M0 or T3 N M0 or T4 N0-N1 M0 |
| Stage IIIB | T1a-T2b N3 M0 or T3-T4 N2 M0 |
| Stage IVA | T (any) N (any) M1a-M1b |
| Stage IVB | T (any) N (any) M1c |

Conventional treatment varies by cell type and by stage of disease. Many patient factors not related to the tumor affect treatment choice. Poor cardiopulmonary reserve, undernutrition, frailty or poor physical performance status, comorbidities, including cytopenias, and psychiatric or cognitive illness all may lead to a decision for palliative over curative treatment or for no treatment at all, even though a cure with aggressive therapy might technically be possible.

SCLC of any stage is typically initially responsive to treatment, but responses are usually short-lived. Chemotherapy, with or without radiation therapy, is given depending on the stage of disease. In many patients, chemotherapy prolongs survival and improves quality of life enough to warrant its use. Surgery generally plays no role in treatment of SCLC, although it may be curative in the rare patient who has a small focal tumor without spread (such as a solitary pulmonary nodule) who underwent surgical resection before the tumor was identified as SCLC. Chemotherapy regimens of etoposide and a platinum compound (either cisplatin or carboplatin) are commonly used, as are other drugs, such as irinotecan, topotecan, vinca alkaloids (vinblastine, vincristine, vinorelbine), alkylating agents (cyclophosphamide, ifosfamide), doxorubicin, taxanes (docetaxel, paclitaxel), and gemcitabine. When disease is confined to a hemithorax, radiation therapy further improves clinical outcomes; such response to radiation therapy was the basis for the definition of limited-stage disease. The use of cranial radiation to prevent brain metastases is also advocated in certain cases; micrometastases are common in SCLC, and chemotherapy has less ability to cross the blood-brain barrier.

### Treatment of Cancer

The invention provides methods for reducing growth of cancer cells, including metastatic cancer cells, through radiation therapy combined with a CD47 blocking agent, e.g. soluble SIRPα monomer or multimer, an anti-CD47 antibody, an anti-SIRPα antibody, small molecule, etc. In certain embodiments the cancer is a lung cancer. In some embodiments the lung cancer is SCLC. In some embodiments the lung cancer is NSCLC. In some embodiments the cancer is metastatic, and an abscopal effect is observed with respect to the area that is irradiated.

“Reducing growth of cancer cells” includes, but is not limited to, reducing proliferation of cancer cells including invasive and/or metastatic cancer cells, and reducing the incidence of a non-cancerous cell becoming a cancerous cell. Whether a reduction in cancer cell growth has been achieved can be readily determined using any known assay, including, but not limited to, [³H]-thymidine incorporation; counting cell number over a period of time; detecting and/or measuring a marker associated with SCLC, etc.

In some embodiments, a combination therapy comprising radiation therapy in combination with CD47 blockade increases an abscopal effect, relative to radiation therapy alone. The abscopal effect can result in a reduction in tumor volume of a non-irradiated tumor mass. In some embodiments, combination therapy comprising radiation therapy in combination with CD47 blockade increases the abscopal effect such that there is about a 10% reduction in tumor volume of a non-irradiated tumor mass, relative to radiation therapy alone. In some embodiments, there is a 10-20%, 20-30%, 30-40%, 40-50%, 50-60%, 60-70%, 70-80%, 80-90%, 90-100%, or greater than 100% reduction in tumor volume of the non-irradiated tumor, relative to radiation therapy alone.

A “therapeutically effective dose” or “therapeutic dose” is an amount sufficient to effect desired clinical results (i.e., achieve therapeutic efficacy). A therapeutically effective dose can be administered in one or more administrations. For purposes of this invention, a therapeutically effective dose of an anti-CD47 agent is an amount that is sufficient to palliate, ameliorate, stabilize, reverse, prevent, slow or delay the progression of the disease state.

The radiation that is administered can be one or more of external beam radiation therapy (EBRT) or brachytherapy (internal radiation therapy). Variations of EBRT include stereotactic body radiation therapy (SBRT) SBRT uses very focused beams of high-dose radiation given in fewer (usually 1 to 5) treatments, where several beams are aimed at the tumor from different angles. Three-dimensional conformal radiation therapy (3D-CRT) uses computers to precisely map the tumor's location. Intensity modulated radiation therapy (IMRT) is a form of 3D therapy. Along with shaping the beams and aiming them at the tumor from several angles, the strength of the beams can be adjusted to limit the dose reaching nearby normal tissues. A variation of IMRT is called volumetric modulated arc therapy (VMAT). It uses a machine that delivers radiation quickly as it rotates once around the body.

A number of fractionation schemes can be used to deliver radiation. Conventionally fractionated (CF, given in 2 Gy once-daily fractions over 6 weeks) can be administered at a dose of from about 10 Gy to about 60 Gy, as is known in the art, e.g. up to about 20 Gy, 30 Gy, 40 Gy, 50 Gy, 60 Gy or more as is known for standard of care. An alternative scheme delivers 66 Gy in 2.75-Gy fractions.

Alternative schemes may use hyperfractionation (lower dose per fraction over the standard treatment duration), accelerated fractionation (conventional fraction size and same total dose, given in a shorter period), accelerated hyperfractionation (combination of these 2), and hypofractionation (higher dose per fraction and fewer fractions).

Specific altered fractionation schemes include 45 Gy/15 fractions (hypofractionation), 69.6 Gy/58 fractions twice daily (BID) (hyperfractionation), 54 Gy/36 fractions TID over 12 consecutive days (continuous hyperfractionated accelerated radiation therapy [CHART], accelerated hyperfractionation), and 60 Gy/40 fractions TID over 18 days (continuous hyperfractionated accelerated radiation therapy weekend-less; thrice-daily (TID) radiation therapy to a dose of 54 Gy in 1.5 Gy per fraction (6-hour intervals over 12 consecutive days).

The CD47 blocking agent may be administered at the same time as radiation, or may be administered on alternative days, before or after delivery of radiation, etc., usually not more than 1, not more than 2, not more than 3 days before or after radiation. Sub-therapeutic CD47 priming dose(s) may be administered prior to radiation, so that a therapeutic dose is appropriately delivered at the time radiation therapy commences.

In some embodiments, additional therapeutic agents are administered in conjunction to the CD47 blocking agent. Therapeutic agents that find use in the present disclosure include, without limitation, immunotherapy, hormone therapy, chemotherapy, etc. When immunotherapy is used an immune checkpoint inhibitor may be administered. Immune checkpoint inhibitors that find use in the present disclosure, include without limitation, CTLA-4 blocking agents, PD-1/PD-L1 blocking agents, LAG-3 blocking agents, etc. In some embodiments, the PD-1/PD-L1 blocking agent is monoclonal anti-PD-1 antibody clone RMP1-14.

Methods are provided for treating a subject with a therapeutic dose of anti-CD47 agent. The subject methods include a step of administering a priming dose to the subject, followed by a step of administering a therapeutically effective dose of an anti-CD47 agent to the subject. In some embodiments, the step of administering a therapeutically effective dose is performed after at least about 3 days (e.g., at least about 4 days, at least about 5 days, at least about 6 days, at least about 7 days, at least about 8 days, at least about 9 days, or at least about 10 days) after beginning the administration of a primer agent. This period of time is, for example, sufficient to provide for enhanced reticulocyte production by the individual.

In some embodiments, a priming dose is defined as a sub-therapeutic dose (i.e., an amount) that is sufficient to cause compensatory reticulocytosis in the recipient, without undue anemia. In some embodiments a priming dose is defined as a dose that causes an anemia that is not worsened by subsequent doses.

The specific appropriate priming dose of an anti-CD47 agent can vary depending on the nature of the agent used and on numerous subject-specific factors (e.g., age, weight, etc.). Examples of suitable priming doses of an anti-CD47 agent include from about 0.5 mg/kg to about 5 mg/kg, from about 0.5 mg/kg to about 4 mg/kg, from about 0.5 mg/kg to about 3 mg/kg, from about 1 mg/kg to about 5 mg/kg, from about 1 mg/kg to about 4 mg/kg, from about 1 mg/kg to about 3 mg/kg, about 1 mg/kg, about 2 mg/kg, about 3 mg/kg, about 4 mg/kg, about 5 mg/kg.

In some embodiments, a therapeutically effective dose leads to sustained serum levels of anti-CD47 agent, i.e. trough levels (e.g., an anti-CD47 antibody) of about 40 μg/ml or more (e.g, about 50 μg/ml or more, about 60 μg/ml or more, about 75 μg/ml or more, about 100 μg/ml or more, about 125 μg/ml or more, or about 150 μg/ml or more). In some embodiments, a therapeutically effective dose leads to sustained serum levels of anti-CD47 agent (e.g., an anti-CD47 antibody) that range from about 40 μg/ml to about 300 μg/ml (e.g, from about 40 μg/ml to about 250 μg/ml, from about 40 μg/ml to about 200 μg/ml, from about 40 μg/ml to about 150 μg/ml, from about 40 μg/ml to about 100 μg/ml, from about 50 μg/ml to about 300 μg/ml, from about 50 μg/ml about 250 μg/ml, from about 50 μg/ml to about 200 μg/ml, from about 50 μg/ml to about 150 μg/ml, from about 75 μg/ml to about 300 μg/ml from about 75 μg/ml to about 250 μg/ml, from about 75 μg/ml to about 200 μg/ml, from about 75 μg/ml to about 150 μg/ml, from about 100 μg/ml about 300 μg/ml, from about 100 μg/ml about 250 μg/ml, or from about 100 μg/ml to about 200 μg/ml). In some embodiments, a therapeutically effective dose for treating solid tumors leads to sustained serum levels of anti-CD47 agent (e.g., an anti-CD47 antibody) of about 100 μg/ml or more, e.g., sustained serum levels that range from about 100 μg/ml to about 500 μg/ml, from about 100 μg/ml to about 400 μg/ml, from about 100 μg/ml to about 300 μg/ml, from about 100 μg/ml to about 200 μg/ml.

Accordingly, series of therapeutically effective doses can achieve and maintain a serum level of anti-CD47 agent. A therapeutically effective dose of an anti-CD47 agent can depend on the specific agent used, but is usually about 5 mg/kg body weight or more (e.g., about 8 mg/kg or more, about 10 mg/kg or more, about 15 mg/kg or more, about 20 mg/kg or more, about 25 mg/kg or more, about 30 mg/kg or more, about 35 mg/kg or more, about 40 mg/kg, about 50 mg/kg or more), or from about 10 mg/kg to about 50 mg/kg (e.g., from about 10 mg/kg to about 40 mg/kg, or from about 10 mg/kg to about 30 mg/kg). The dose required to achieve and/or maintain a particular serum level is proportional to the amount of time between doses and inversely proportional to the number of doses administered. Thus, as the frequency of dosing increases, the required dose decreases. The optimization of dosing strategies will be readily understood and practiced by one of ordinary skill in the art.

A “maintenance dose” is a dose intended to be a therapeutically effective dose. For example, in experiments to determine the therapeutically effective dose, multiple different maintenance doses may be administered to different subjects. As such, some of the maintenance doses may be therapeutically effective doses and others may be sub-therapeutic doses.

A “loading dose” may be used to achieve a therapeutic level of antibody before switching to a maintenance dose. A loading dose can be the same be the same or higher or lower than the maintenance dose, but will generally provide for a higher overall delivery of the agent over a given period of time. For example, a loading dose can be the same or lower than a maintenance dose, but delivered more frequently, e.g. daily, every other day, every third day, twice weekly, weekly, and the like. Alternatively a loading dose can be a higher dose than a maintenance dose, and delivered at the same periodicity, or more frequently, e.g. daily, every other day, every third day, twice weekly, weekly, and the like.

The administration of a therapeutically effective dose of an anti-CD47 agent can be achieved in a number of different ways. In some cases, two or more therapeutically effective doses are administered after a primer agent is administered. Suitable administration of a therapeutically effective dose can entail administration of a single dose, or can entail administration of doses daily, semi-weekly, weekly, once every two weeks, once a month, annually, etc. In some cases, a therapeutically effective dose is administered as two or more doses of escalating concentration (i.e., increasing doses), where (i) all of the doses are therapeutic doses, or where (ii) a sub-therapeutic dose (or two or more sub-therapeutic doses) is initially given and therapeutic doses are achieved by said escalation. As one non-limiting example to illustrate escalating concentration (i.e., increasing doses), a therapeutically effective dose can be administered weekly, beginning with a sub-therapeutic dose (e.g., a dose of 5 mg/kg), and each subsequent dose can be increased by a particular increment (e.g., by 5 mg/kg), or by variable increments, until a therapeutic dose (e.g., 30 mg/kg) is reached, at which point administration may cease or may continue (e.g., continued therapeutic doses, e.g., doses of 30 mg/kg). As another non-limiting example to illustrate escalating concentration (i.e., increasing doses), a therapeutically effective dose can be administered weekly, beginning with a therapeutic dose (e.g., a dose of 10 mg/kg), and each subsequent dose can be increased by a particular increment (e.g., by 10 mg/kg), or by variable increments, until a therapeutic dose (e.g., 30 mg/kg, 100 mg/ml, etc.) is reached, at which point administration may cease or may continue (e.g., continued therapeutic doses, e.g., doses of 30 mg/kg, 100 mg/ml, etc.). In some embodiments, administration of a therapeutically effective dose can be a continuous infusion and the dose can be altered (e.g., escalated) over time.

Dosage and frequency may vary depending on the half-life of the anti-CD47 agent in the patient. It will be understood by one of skill in the art that such guidelines will be adjusted for the molecular weight of the active agent, e.g. in the use of antibody fragments, in the use of antibody conjugates, in the use of SIRPα reagents, in the use of soluble CD47 peptides etc. The dosage may also be varied for localized administration, e.g. intranasal, inhalation, etc., or for systemic administration, e.g. i.m., i.p., i.v., s.c., and the like.

An initial dose of a CD47 binding agent, including but not limited to a priming dose, may lead to hemagglutination for a period of time immediately following infusion. Without being bound by the theory, it is believed that the initial dose of a multivalent CD47 binding agent may cause cross-linking of RBC bound to the agent. In certain embodiments of the invention, a CD47 binding agent is infused to a patient in an initial dose, and optionally in subsequent doses, over a period of time and/or concentration that reduces the possibility of hematologic microenvironments where there is a high local concentration of RBC and the agent.

In some embodiments of the invention, an initial dose of a CD47 binding agent is infused over a period of at least about 2 hours, at least about 2.5 hours, at least about 3 hours, at least about 3.5 hours, at least about 4 hours, at least about 4.5 hours, at least about 5 hours, at least about 6 hours or more. In some embodiments an initial dose is infused over a period of time from about 2.5 hours to about 6 hours; for example from about 3 hours to about 4 hours. In some such embodiments, the dose of agent in the infusate is from about 0.05 mg/ml to about 0.5 mg/ml; for example from about 0.1 mg/ml to about 0.25 mg/ml.

In other embodiments, an initial dose of a CD47 binding agent, e.g. a priming dose, is administered by continuous fusion, e.g. as an osmotic pump, delivery patch, etc., where the dose is administered over a period of at least about 6 hours, at least about 12 hours, at least about 24 hours, at least about 2 days, at least about 3 days. Many such systems are known in the art. For example DUROS technology, provides a bi-compartment system separated by a piston. One of the compartments consists of osmotic engine specifically formulated with an excess of solid NaCl, such that it remains present throughout the delivery period and results in a constant osmotic gradient. It also consists of a semi permeable membrane on one end through which water is drawn into the osmotic engine and establishes a large and constant osmotic gradient between the tissue water and the osmotic engine. Another compartment consists of a drug solution with an orifice from which the drug is released due to the osmotic gradient. This helps to provide site specific and systemic drug delivery when implanted in humans. The preferred site of implantation is subcutaneous placement in the inside of the upper arm.

Following administration of the priming agent, and allowing a period of time effective for an increase in reticulocyte production, a therapeutic dose of an anti-CD47 agent is administered. The therapeutic dose can be administered in number of different ways. In some embodiments, two or more therapeutically effective doses are administered after a primer agent is administered, e.g. in a weekly dosing schedule. In some embodiments a therapeutically effective dose of an anti-CD47 agent is administered as two or more doses of escalating concentration, in others the doses are equivalent. There is reduced hemagglutination after the priming dose, and therefore the extended infusion time is not required.

The therapeutic agents, e.g. a CD47 blocking agent, is formulated with a non-toxic, pharmaceutically acceptable carrier substance. Usually, this will be an aqueous solution, such as normal saline or phosphate-buffered saline (PBS), Ringer's solution, lactate-Ringer's solution, or any isotonic physiologically acceptable solution for administration by the chosen means. Preferably, the solution is sterile and pyrogen-free, and is manufactured and packaged under current Good Manufacturing Processes (GMPs), as approved by the FDA. The clinician of ordinary skill is familiar with appropriate ranges for pH, tonicity, and additives or preservatives when formulating pharmaceutical compositions for administration by intravascular injection, direct injection into the lymph nodes, intraperitoneal, or by other routes. In addition to additives for adjusting pH or tonicity, the agents may be stabilized against aggregation and polymerization with amino acids and non-ionic detergents, polysorbate, and polyethylene glycol. Optionally, additional stabilizers may include various physiologically-acceptable carbohydrates and salts. Also, polyvinylpyrrolidone may be added in addition to the amino acid. Suitable therapeutic immunoglobulin solutions which are stabilized for storage and administration to humans are described in U.S. Pat. No. 5,945,098, incorporated fully herein by reference. Other agents, such as human serum albumin (HSA), may be added to the therapeutic or imaging composition to stabilize the antibody conjugates.

The compositions of the invention may be administered using any medically appropriate procedure, e.g., intravascular (intravenous, intraarterial, intracapillary) administration, injection into the tumor, etc. Intravascular injection may be by intravenous or intraarterial injection. The effective amount of the therapeutic compositions to be given to a particular patient will depend on a variety of factors, several of which will be different from patient to patient. A competent clinician will be able to determine an effective amount of a therapeutic composition to administer to a patient to retard the growth and promote the death of tumor cells. Dosage of the agents will depend on the treatment of the tumor, route of administration, the nature of the therapeutics, sensitivity of the tumor to the therapeutics, etc. Utilizing LD₅₀ animal data, and other information available for the conjugated cytotoxic or imaging moiety, a clinician can determine the maximum safe dose for an individual, depending on the route of administration. For instance, an intravenously administered dose may be more than a locally administered dose, given the greater body of fluid into which the therapeutic composition is being administered. Similarly, compositions which are rapidly cleared from the body may be administered at higher doses, or in repeated doses, in order to maintain a therapeutic concentration. Utilizing ordinary skill, the competent clinician will be able to optimize the dosage of a particular therapeutic or imaging composition in the course of routine clinical trials.

The combination treatment methods disclosed herein may be used alone or in combination with other therapeutic intervention such as chemotherapy, immunosuppressant and immunomodulatory therapies, cell therapy, and transplantation.

Chemotherapy may include Abitrexate (Methotrexate Injection), Abraxane (Paclitaxel Injection), Adcetris (Brentuximab Vedotin Injection), Adriamycin (Doxorubicin), Adrucil Injection (5-FU (fluorouracil)), Afinitor (Everolimus), Afinitor Disperz (Everolimus), Alimta (PEMET EXED), Alkeran Injection (Melphalan Injection), Alkeran Tablets (Melphalan), Aredia (Pamidronate), Arimidex (Anastrozole), Aromasin (Exemestane), Arranon (Nelarabine), Arzerra (Ofatumumab Injection), Avastin (Bevacizumab), Bexxar (Tositumomab), BiCNU (Carmustine), Blenoxane (Bleomycin), Bosulif (Bosutinib), Busulfex Injection (Busulfan Injection), Campath (Alemtuzumab), Camptosar (Irinotecan), Caprelsa (Vandetanib), Casodex (Bicalutamide), CeeNU (Lomustine), CeeNU Dose Pack (Lomustine), Cerubidine (Daunorubicin), Clolar (Clofarabine Injection), Cometriq (Cabozantinib), Cosmegen (Dactinomycin), CytosarU (Cytarabine), Cytoxan (Cytoxan), Cytoxan Injection (Cyclophosphamide Injection), Dacogen (Decitabine), DaunoXome (Daunorubicin Lipid Complex Injection), Decadron (Dexamethasone), DepoCyt (Cytarabine Lipid Complex Injection), Dexamethasone Intensol (Dexamethasone), Dexpak Taperpak (Dexamethasone), Docefrez (Docetaxel), Doxil (Doxorubicin Lipid Complex Injection), Droxia (Hydroxyurea), DTIC (Decarbazine), Eligard (Leuprolide), Ellence (Ellence (epirubicin)), Eloxatin (Eloxatin (oxaliplatin)), Elspar (Asparaginase), Emcyt (Estramustine), Erbitux (Cetuximab), Erivedge (Vismodegib), Erwinaze (Asparaginase Erwinia chrysanthemi), Ethyol (Amifostine), Etopophos (Etoposide Injection), Eulexin (Flutamide), Fareston (Toremifene), Faslodex (Fulvestrant), Femara (Letrozole), Firmagon (Degarelix Injection), Fludara (Fludarabine), Folex (Methotrexate Injection), Folotyn (Pralatrexate Injection), FUDR (FUDR (floxuridine)), Gemzar (Gemcitabine), Gilotrif (Afatinib), Gleevec (Imatinib Mesylate), Gliadel Wafer (Carmustine wafer), Halaven (Eribulin Injection), Herceptin (Trastuzumab), Hexalen (Altretamine), Hycamtin (Topotecan), Hycamtin (Topotecan), Hydrea (Hydroxyurea), Iclusig (Ponatinib), Idamycin PFS (Idarubicin), Ifex (Ifosfamide), Inlyta (Axitinib), Intron A alfab (Interferon alfa-2a), Iressa (Gefitinib), Istodax (Romidepsin Injection), Ixempra (Ixabepilone Injection), Jakafi (Ruxolitinib), Jevtana (Cabazitaxel Injection), Kadcyla (Ado-trastuzumab Emtansine), Kyprolis (Carfilzomib), Leukeran (Chlorambucil), Leukine (Sargramostim), Leustatin (Cladribine), Lupron (Leuprolide), Lupron Depot (Leuprolide), Lupron DepotPED (Leuprolide), Lysodren (Mitotane), Marclibo Kit (Vincristine Lipid Complex Injection), Matulane (Procarbazine), Megace (Megestrol), Mekinist (Trametinib), Mesnex (Mesna), Mesnex (Mesna Injection), Metastron (Strontium-89 Chloride), Mexate (Methotrexate Injection), Mustargen (Mechlorethamine), Mutamycin (Mitomycin), Myleran (Busulfan), Mylotarg (Gemtuzumab Ozogamicin), Navelbine (Vinorelbine), Neosar Injection (Cyclophosphamide Injection), Neulasta (filgrastim), Neulasta (pegfilgrastim), Neupogen (filgrastim), Nexavar (Sorafenib), Nilandron (Nilandron (nilutamide)), Nipent (Pentostatin), Nolvadex (Tamoxifen), Novantrone (Mitoxantrone), Oncaspar (Pegaspargase), Oncovin (Vincristine), Ontak (Denileukin Diftitox), Onxol (Paclitaxel Injection), Panretin (Alitretinoin), Paraplatin (Carboplatin), Perjeta (Pertuzumab Injection), Platinol (Cisplatin), Platinol (Cisplatin Injection), PlatinolAQ (Cisplatin), PlatinolAQ (Cisplatin Injection), Pomalyst (Pomalidomide), Prednisone Intensol (Prednisone), Proleukin (Aldesleukin), Purinethol (Mercaptopurine), Reclast (Zoledronic acid), Revlimid (Lenalidomide), Rheumatrex (Methotrexate), Rituxan (Rituximab), RoferonA alfaa (Interferon alfa-2a), Rubex (Doxorubicin), Sandostatin (Octreotide), Sandostatin LAR Depot (Octreotide), Soltamox (Tamoxifen), Sprycel (Dasatinib), Sterapred (Prednisone), Sterapred DS (Prednisone), Stivarga (Regorafenib), Supprelin LA (Histrelin Implant), Sutent (Sunitinib), Sylatron (Peginterferon Alfa-2b Injection (Sylatron)), Synribo (Omacetaxine Injection), Tabloid (Thioguanine), Taflinar (Dabrafenib), Tarceva (Erlotinib), Targretin Capsules (Bexarotene), Tasigna (Decarbazine), Taxol (Paclitaxel Injection), Taxotere (Docetaxel), Temodar (Temozolomide), Temodar (Temozolomide Injection), Tepadina (Thiotepa), Thalomid (Thalidomide), TheraCys BCG (BCG), Thioplex (Thiotepa), TICE BCG (BCG), Toposar (Etoposide Injection), Torisel (Temsirolimus), Treanda (Bendamustine hydrochloride), Trelstar (Triptorelin Injection), Trexall (Methotrexate), Trisenox (Arsenic trioxide), Tykerb (lapatinib), Valstar (Valrubicin Intravesical), Vantas (Histrelin Implant), Vectibix (Panitumumab), Velban (Vinblastine), Velcade (Bortezomib), Vepesid (Etoposide), Vepesid (Etoposide Injection), Vesanoid (Tretinoin), Vidaza (Azacitidine), Vincasar PFS (Vincristine), Vincrex (Vincristine), Votrient (Pazopanib), Vumon (Teniposide), Wellcovorin IV (Leucovorin Injection), Xalkori (Crizotinib), Xeloda (Capecitabine), Xtandi (Enzalutamide), Yervoy (Ipilimumab Injection), Zaltrap (Ziv-aflibercept Injection), Zanosar (Streptozocin), Zelboraf (Vemurafenib), Zevalin (Ibritumomab Tiuxetan), Zoladex (Goserelin), Zolinza (Vorinostat), Zometa (Zoledronic acid), Zortress (Everolimus), Zytiga (Abiraterone), Nimotuzumab and immune checkpoint inhibitors such as nivolumab, pembrolizumab/MK-3475, pidilizumab and AMP-224 targeting PD-1; and BMS-935559, MEDI4736, MPDL3280A and MSB0010718C targeting PD-L1 and those targeting CTLA-4 such as ipilimumab.

The invention now being fully described, it will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### EXAMPLE 1

### Macrophage-Mediated Abscopal Effects of Radiation Therapy

Radiation therapy is widely used to treat cancer, but many patients still suffer from local recurrence and/or metastatic disease following radiation. Observations of innate and adaptive immune responses following radiation has led to the idea that combining radiation therapy and immunotherapy may provide benefits to cancer patients, both locally and systemically. Here we combine radiation therapy with activation of macrophage-mediated phagocytosis via CD47 blockade in small cell lung cancer (SCLC), an aggressive form of lung cancer. This combination therapy promotes potent local antitumor effects in pre-clinical models of SCLC. Strikingly, CD47 blockade also stimulates abscopal anti-SCLC effects in a T-cell-independent and macrophage-dependent manner. The systemic activation of antitumor macrophages following radiation therapy is particularly important in cancer patients who suffer from metastatic disease.

Small cell lung cancer (SCLC) is a fatal form of lung cancer with high rates of metastasis. In SCLC patients, radiation therapy is often combined with chemotherapy and is used with both curative and palliative intent. However, SCLC tumors nearly always relapse quickly, and the survival rate of SCLC patients remains very low. Recently, immunotherapies focusing on inhibition of PD-1/PD-L1 and subsequent activation of T cells have been approved for the treatment of SCLC. Unfortunately, these new approaches only extend overall survival by ˜2 months, and there is still a great unmet need to develop therapeutic approaches that improve the survival of SCLC patients. We previously found that the “don't-eat-me” molecule CD47 is highly expressed on the surface of SCLC cells and that blockade of CD47 can enhance the phagocytosis of SCLC cells by macrophages. Here we asked whether combining CD47 blockade and radiation therapy could synergize to stunt the expansion of SCLC tumors in murine and human pre-clinical models of SCLC. We made the surprising observation that CD47 blockade not only potentiates local antitumor effects of radiation therapy but also stimulates abscopal antitumor effects.

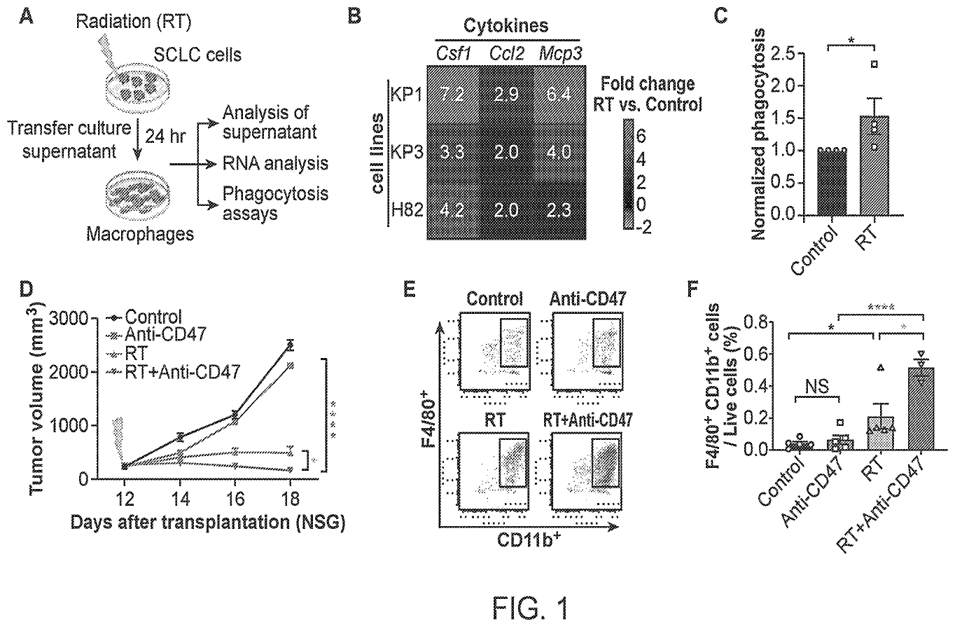

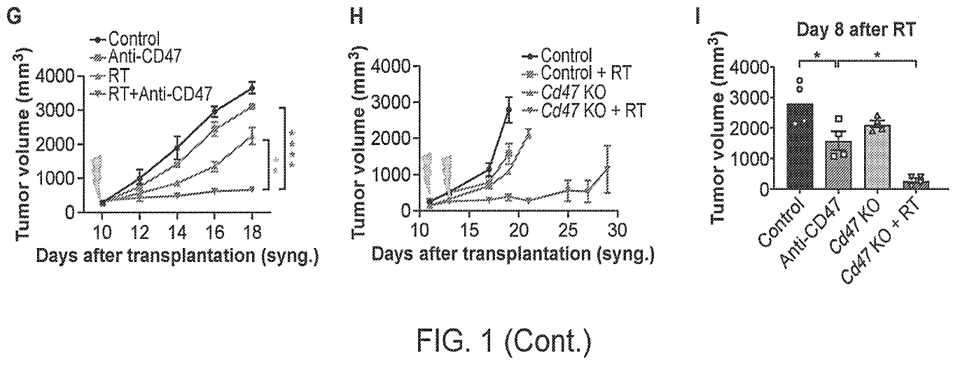

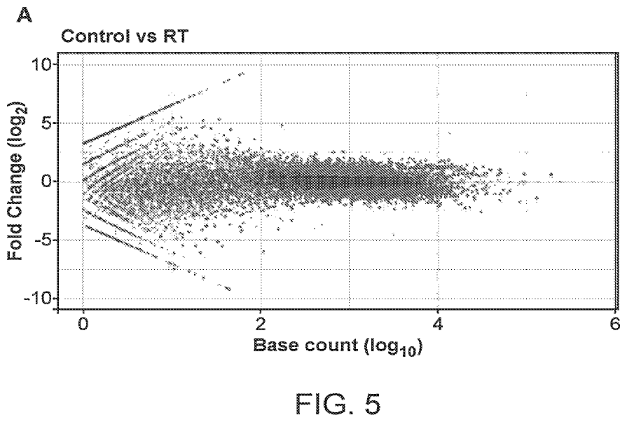

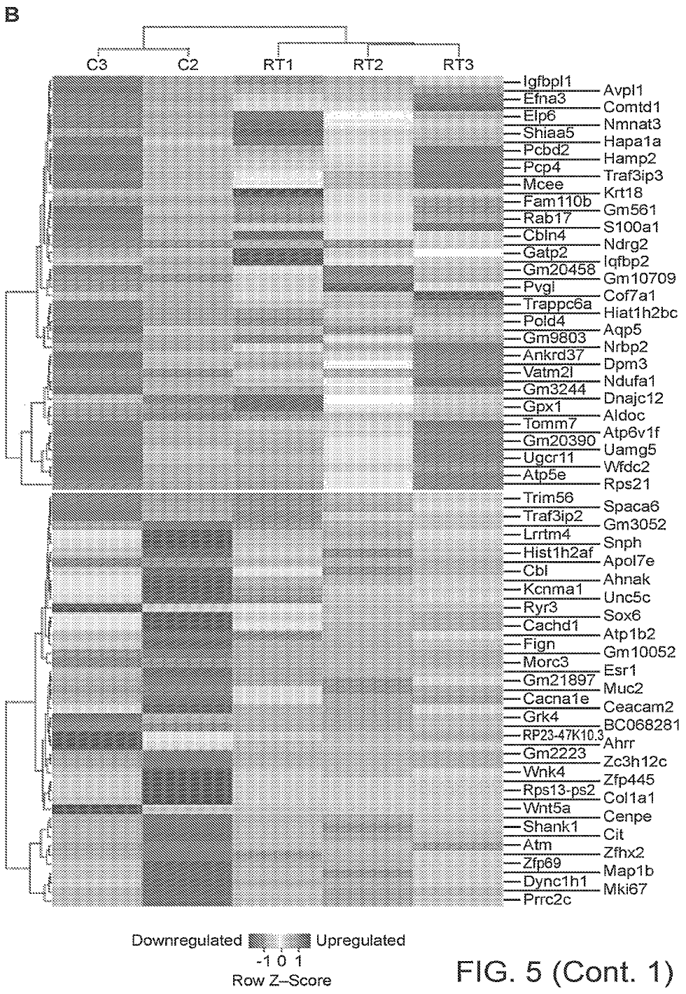

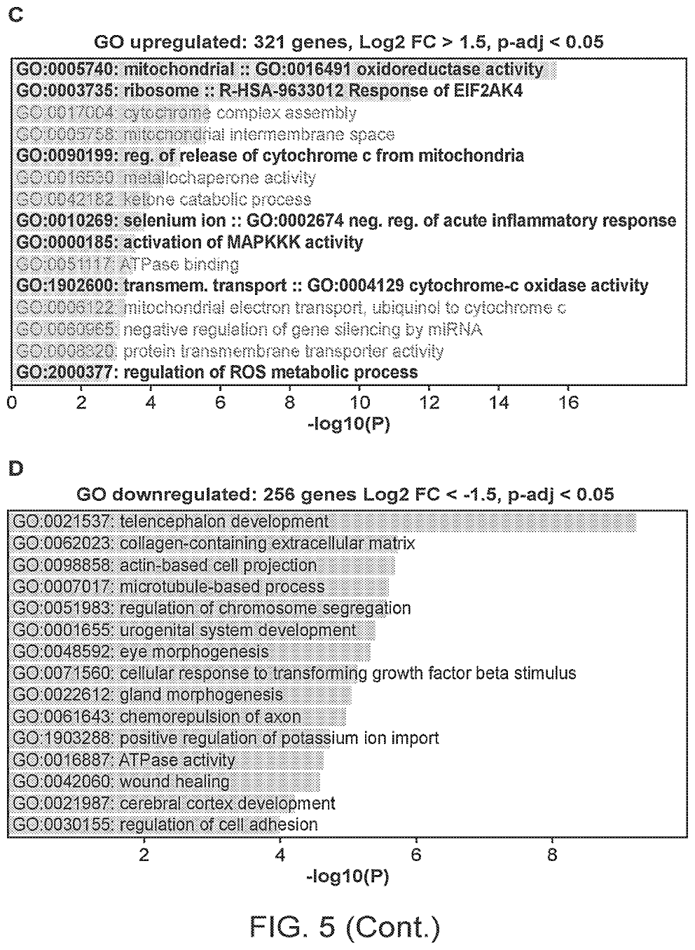

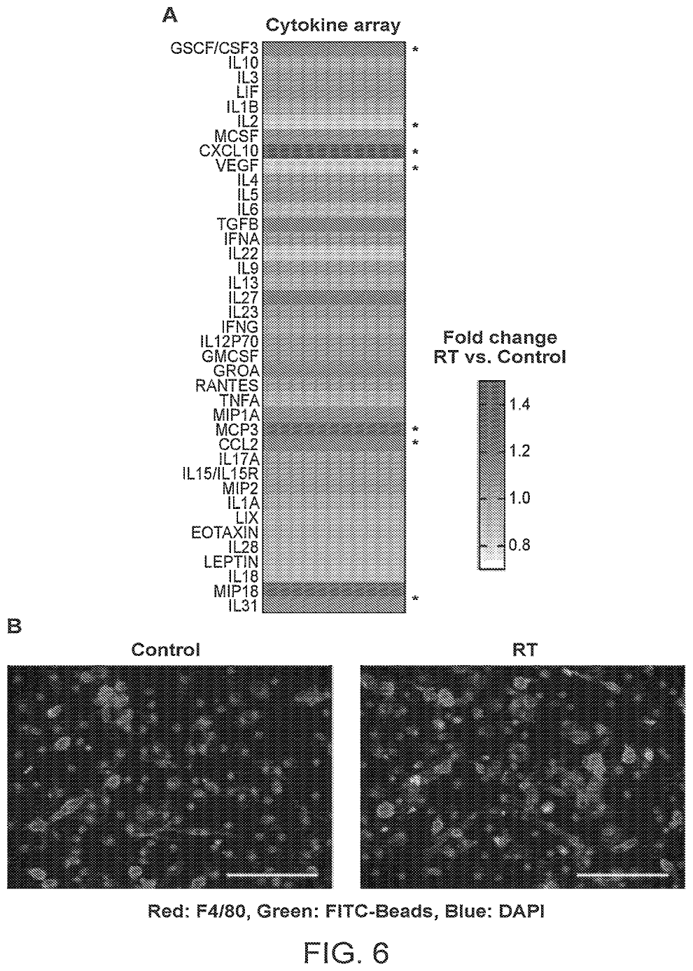

We first evaluated the transcriptional response of SCLC cells to radiation by bulk RNA sequencing (FIGS. 1A, 5A-D). We identified 321 upregulated and 256 downregulated genes with corrected p-value <0.05 and log2 fold change of >1.5 and <−1.5. Gene Ontology (GO) biological processes for the top genes induced 24 hours after radiation were enriched for processes related to inflammation and stress response while genes down-regulated by radiation were enriched in developmental and cell division processes (FIGS. 5A-D). To further examine how stress signals and inflammation induced by radiation may recruit and activate macrophages, we used a cytokine array to query the supernatant of control or irradiated mouse SCLC cells (FIG. 1A). This approach identified an increased secretion of cytokines known to recruit and activate macrophages, including CSF-1, CCL2, and MCP3 (also known as CCL7) (FIG. 6A and Table 2). These results were validated at the RNA level in the same cell line, a second mouse cell line, and a human cell line (FIG. 1B). Accordingly, we found that the supernatant of irradiated mouse SCLC cells enhances the phagocytic ability of mouse bone marrow-derived macrophages ex vivo (FIG. 1C and FIGS. 6B-D) and their migratory ability (FIG. 6E). These experiments in culture suggested that irradiation of SCLC cells may activate and recruit macrophages, encouraging combination of this therapy with CD47 blockade.

TABLE 1
Table 1: Cytokine array data from
control and irradiated SCLC cells

| | RT−_1 | RT−_2 | RT−_3 | RT+_1 | RT+_2 | RT+_3 |
|---|---|---|---|---|---|---|
| GSCF/CSF3 | 42.5 | 39.5 | 41.75 | 46.25 | 47.75 | 45.25 |
| IL10 | 49.5 | 46 | 48 | 48 | 42.75 | 49.25 |
| IL3 | 23.25 | 18 | 17 | 19.75 | 19.5 | 21.25 |
| LIF | 30.25 | 29.75 | 26.5 | 29.5 | 31.25 | 30.5 |
| IL1B | 14.75 | 13 | 15.5 | 14.25 | 13.75 | 14.5 |
| IL2 | 52 | 47 | 46 | 42.5 | 37 | 40.75 |
| MCSF | 62.25 | 58 | 53 | 60.5 | 58.5 | 65.25 |
| IP10 | 3406.5 | 3608.5 | 3320.5 | 5323.5 | 4821 | 5203.25 |
| VEGF | 7635.75 | 7900 | 7906.5 | 6692.5 | 6026 | 6687.25 |
| IL4 | 28 | 31.25 | 26.75 | 26.75 | 29.75 | 31 |
| IL5 | 18 | 20 | 20 | 22.75 | 18.75 | 20 |
| IL6 | 33.75 | 29 | 32 | 24.5 | 33.5 | 28.25 |
| TGFB | 125.25 | 129.5 | 126.75 | 139.25 | 140 | 154.5 |
| IFNA | 323 | 323 | 312.5 | 319.25 | 337.25 | 326.25 |
| IL22 | 18.75 | 21.75 | 18 | 16.75 | 16.5 | 16.25 |
| IL9 | 41.75 | 41.75 | 44 | 48.25 | 41 | 44.75 |
| IL13 | 71.75 | 75.5 | 76 | 71 | 72 | 70.5 |
| IL27 | 13 | 13.5 | 10.75 | 14.25 | 14.5 | 14.75 |
| IL23 | 21.25 | 23.5 | 22.5 | 22.5 | 23.75 | 23.25 |
| IFNG | 33 | 35.75 | 35.25 | 34 | 36.5 | 32.25 |
| IL12P70 | 181 | 179.25 | 153.75 | 156.5 | 174.5 | 208.75 |
| GMCSF | 27 | 25.75 | 24 | 27 | 24.5 | 29 |
| GROA | 41.5 | 39.5 | 36 | 45.75 | 42.25 | 43.75 |
| RANTES | 214.75 | 244.25 | 215 | 230.5 | 234.5 | 219.25 |
| TNFA | 62.25 | 58.5 | 59.75 | 53.5 | 51.75 | 63.75 |
| MIP1A | 157.5 | 160.75 | 146.5 | 159.25 | 175 | 165.5 |
| MCP3 | 50.5 | 55 | 46.5 | 66 | 61.5 | 63.5 |
| MCP1 | 56 | 53 | 53.5 | 64 | 59.5 | 59.75 |
| IL17A | 17.75 | 19.5 | 21.25 | 19 | 19.5 | 20.5 |
| IL15/IL15R | 51.25 | 48 | 48.75 | 51 | 50 | 48.5 |
| MIP2 | 19.5 | 21.5 | 20.5 | 21.5 | 20.25 | 22.5 |
| IL1A | 19 | 20.25 | 16.5 | 17.25 | 17.25 | 19.5 |
| LIX | 28.5 | 27 | 25 | 25 | 23.5 | 26.5 |
| EOTAXIN | 39.25 | 40.5 | 36 | 35.25 | 36.5 | 39.25 |
| IL28 | 24.25 | 25.5 | 27.5 | 23.75 | 23.25 | 24 |
| LEPTIN | 114.5 | 101 | 106.5 | 100.5 | 96.5 | 97.75 |
| IL18 | 52.5 | 56.25 | 49.25 | 49 | 46.25 | 47 |
| MIP1B | 65.75 | 61.5 | 54.25 | 78 | 78 | 78 |
| IL31 | 52 | 50.25 | 45.25 | 55 | 52 | 55 |

Raw values from the experiments with mouse SCLC KP1 cells are shown (RT−, unirradiated; RT+, irradiated)

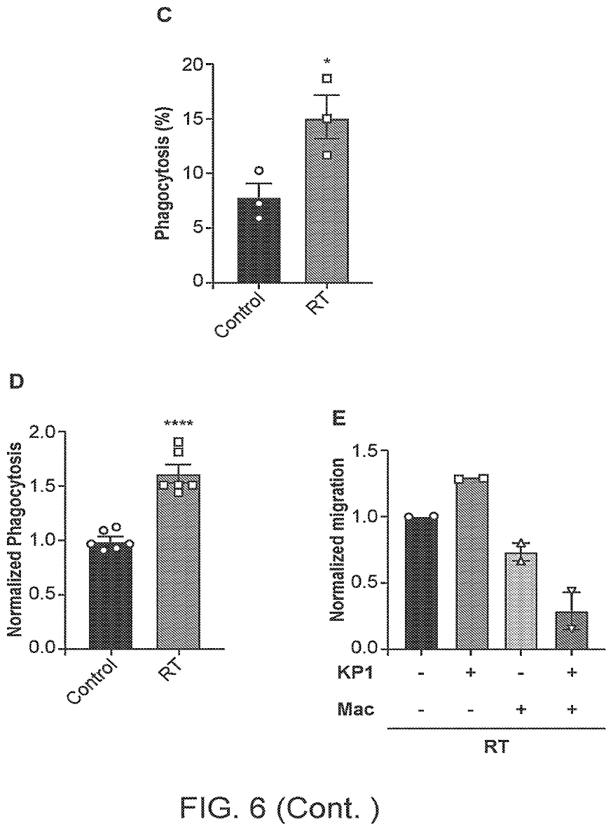

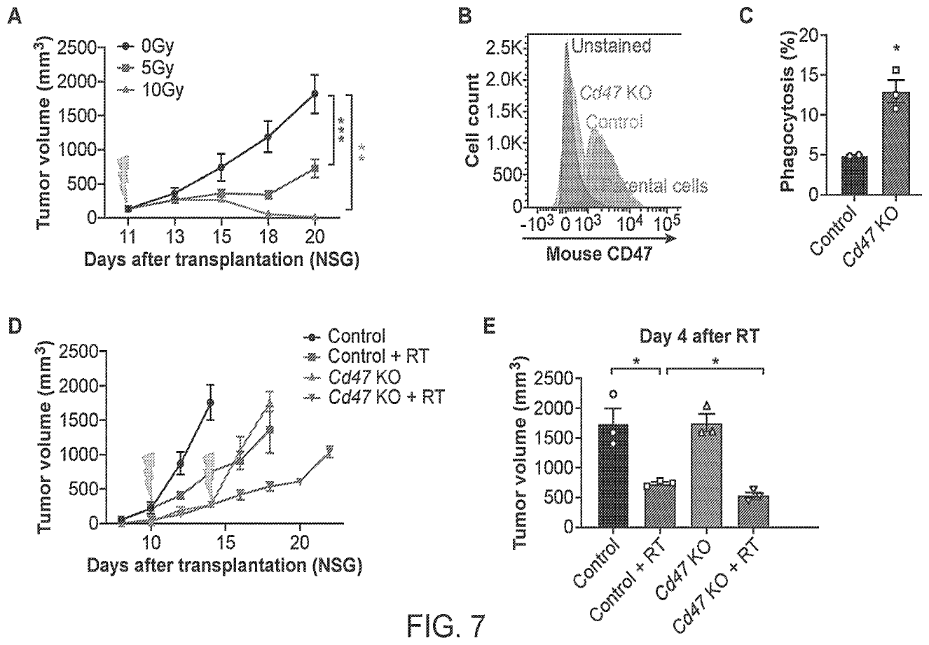

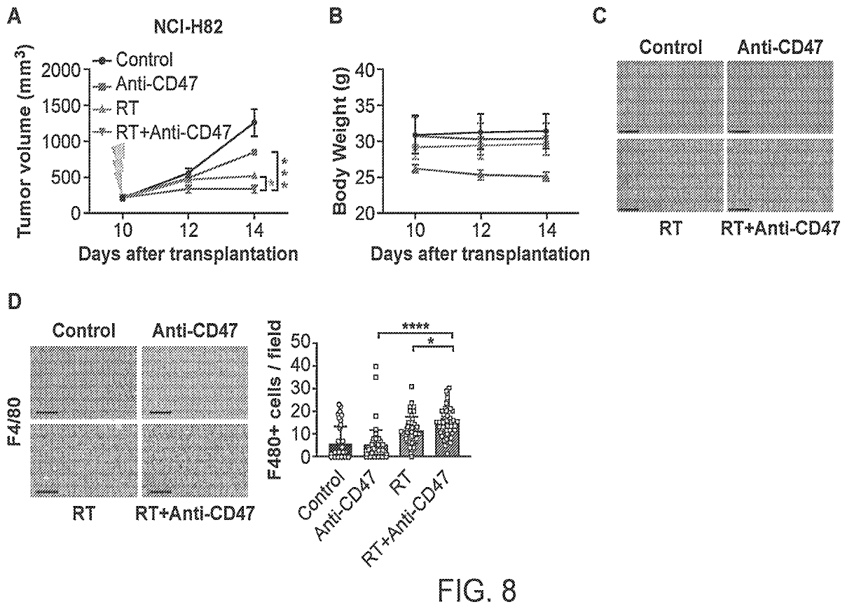

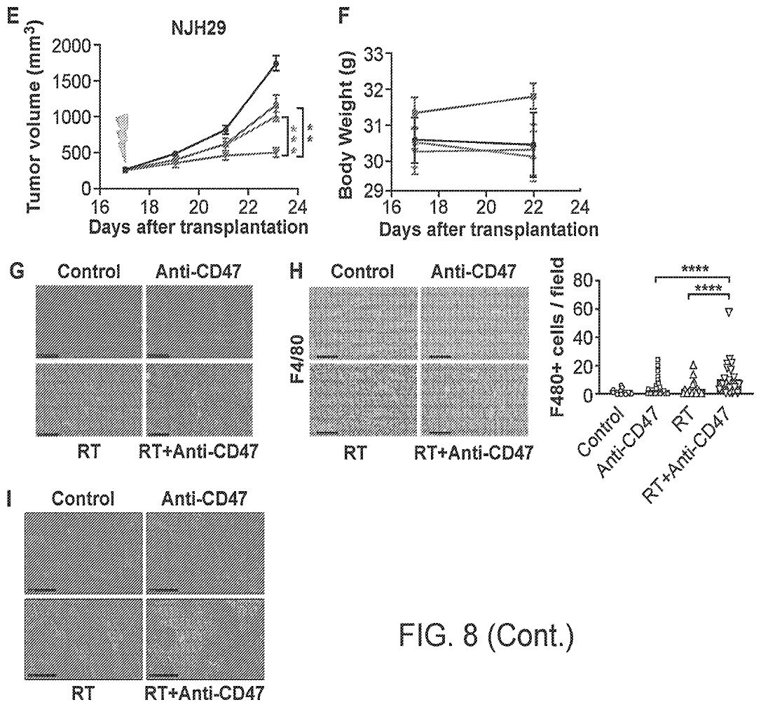

To examine the effects of radiation therapy on SCLC tumors in vivo, mouse SCLC cells were engrafted subcutaneously into the flank of NSG mice, which lack functional T cells, B cells, and NK cells but retain functional monocytes and macrophages. Tumor-bearing mice were treated with different doses of radiation. In these experiments, 5 Gy irradiation inhibited tumor growth while 10 Gy almost eradicated the tumors (FIG. 7A). Based on this result, we decided to use a single fraction dose of 5 Gy in subsequent experiments to be able to identify additional antitumor effects of combination therapies. Irradiation led to inhibition of tumor growth (FIG. 1D) and recruitment of macrophages to the tumor microenvironment (FIGS. 1E-F), confirming the observations in culture. We also combined tumor irradiation with a blocking antibody directed against mouse CD47, as a way to potentiate possible antitumor effects of macrophages activated by radiation therapy. As a single agent, treatment with the CD47-blocking antibody had minimal effects on tumor growth, possibly because of cross-reactivity to CD47 expressed on red blood cells and other cells in the body, with fewer molecules of antibody being able to bind to SCLC cells. In contrast, the combination treatment with radiation therapy significantly inhibited tumor growth (FIG. 1D), which correlated with a further increase in the infiltration of macrophages (FIGS. 1E-F). We performed similar experiments with the same mouse SCLC cell line in which the gene coding for CD47 was knocked out (FIGS. 7B-C). CD47 loss in this context led to decreased tumor growth, which was further reduced by radiation therapy (FIGS. 7D-E). Radiation therapy combined with a blocking antibody directed against human CD47 also had enhanced antitumor effects with two human xenograft models, with increased macrophage infiltration and no significant body weight loss (FIGS. 8A-H). Finally, pharmacologic or genetic CD47 inhibition in subcutaneous mouse SCLC tumors growing in immunocompetent mice still led to enhanced tumor inhibition following radiation, indicating that the effects observed are not prevented by T cells, B cells, or NK cells present in this context (FIG. 1G-I and FIG. 8I). Thus, CD47 inhibition enhances the antitumor effects of radiation in pre-clinical models of SCLC.

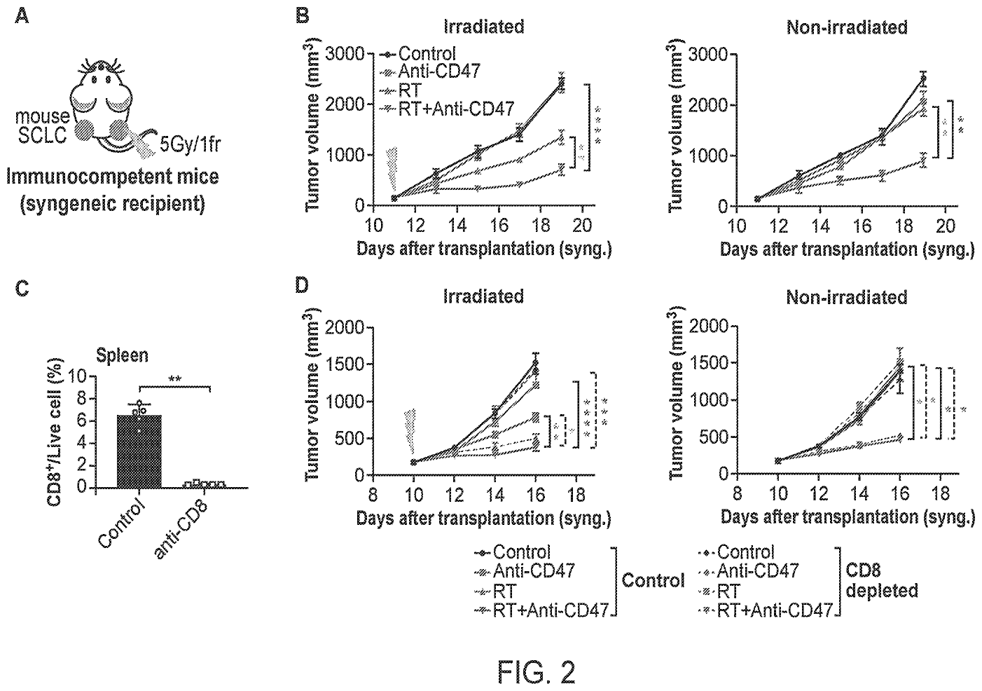

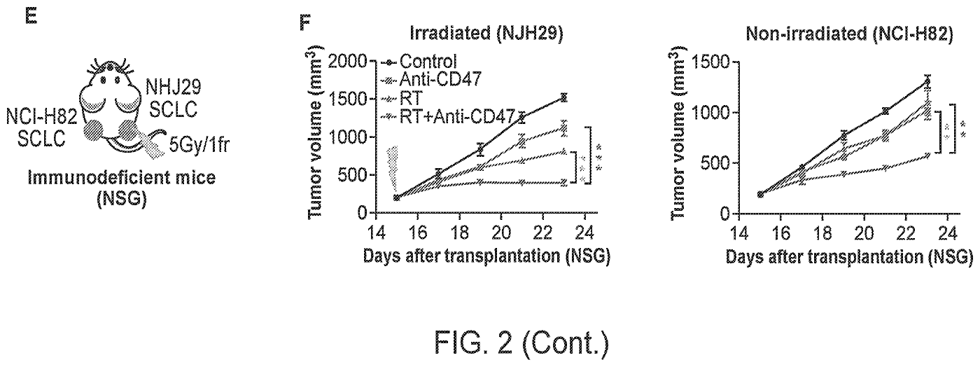

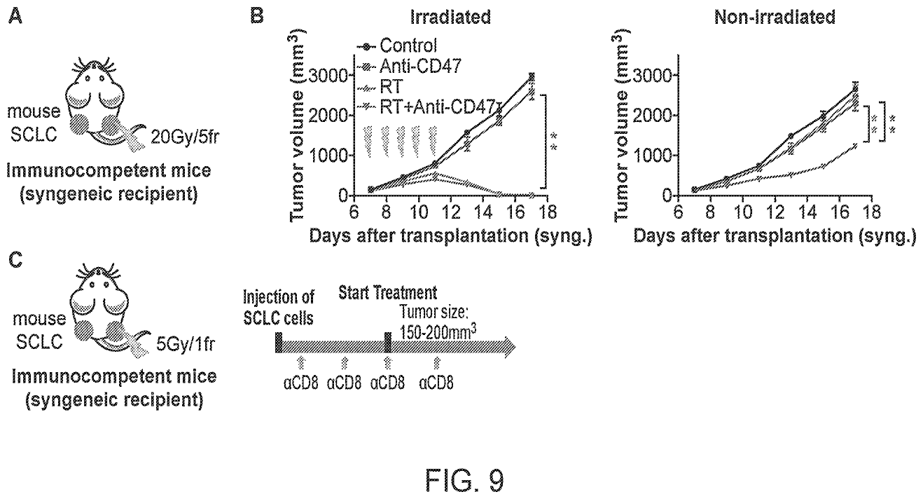

While the local effects of radiation therapy have been investigated in great detail, there is emerging interest in its potential systemic effects. In particular, clinical case reports have described so-called abscopal effects of radiation therapy, where irradiation of a tumor mass results in antitumor effects on another unirradiated tumor lesion in the same individual. The mechanisms mediating this so-called abscopal effect remain poorly understood. To evaluate whether inhibition of CD47 could facilitate abscopal responses to radiotherapy, we first engrafted mouse SCLC cells into the flanks of immunocompetent recipient mice and allowed tumors to grow for ˜2 weeks. Radiation therapy was delivered only to the tumor on the right side of each mouse, with or without concurrent anti-CD47 antibody treatment (FIG. 2A). Similar to mice with just one tumor, radiation therapy and CD47 blockade had an enhanced inhibitory effect on the irradiated tumors (FIG. 2B, left). Importantly, mice treated with the combination therapy had significantly smaller unirradiated tumors compared to mice treated with either treatment alone (FIG. 2B, right). Similar results were observed using a fractionated radiation therapy schedule (20 Gy in 5 fractions) that more closely resembles the regimen SCLC patients undergo in the clinic and that nearly completely eliminated the irradiated tumor (FIGS. 9A-B). In these settings, the abscopal effect was only visible in the combination therapy and not with radiation therapy alone, indicating that CD47 blockade is required to observe this systemic antitumor effect of radiation.

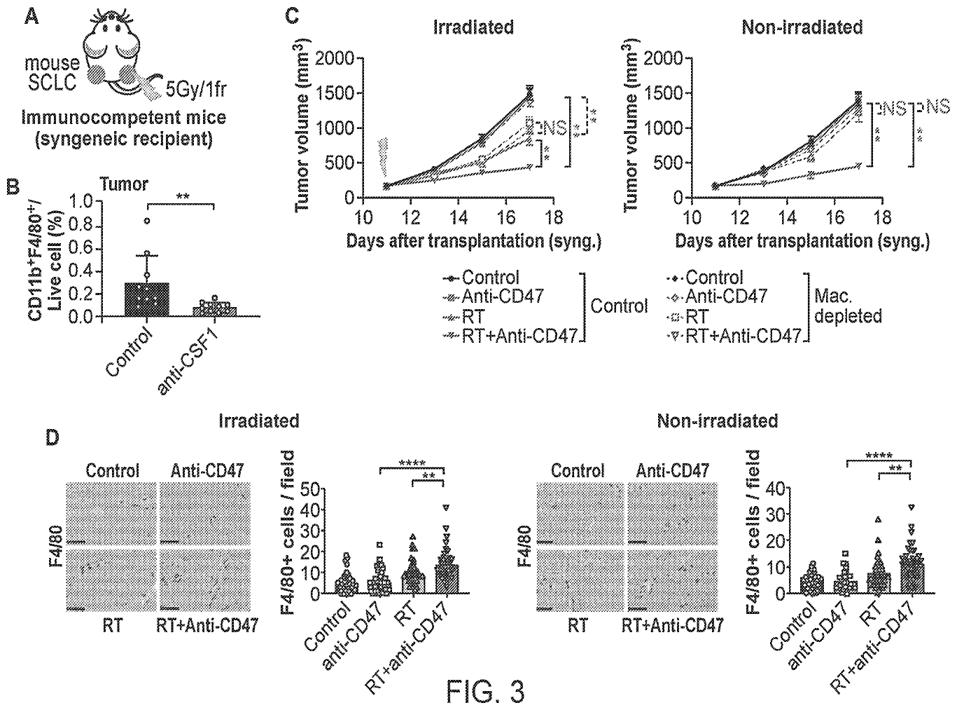

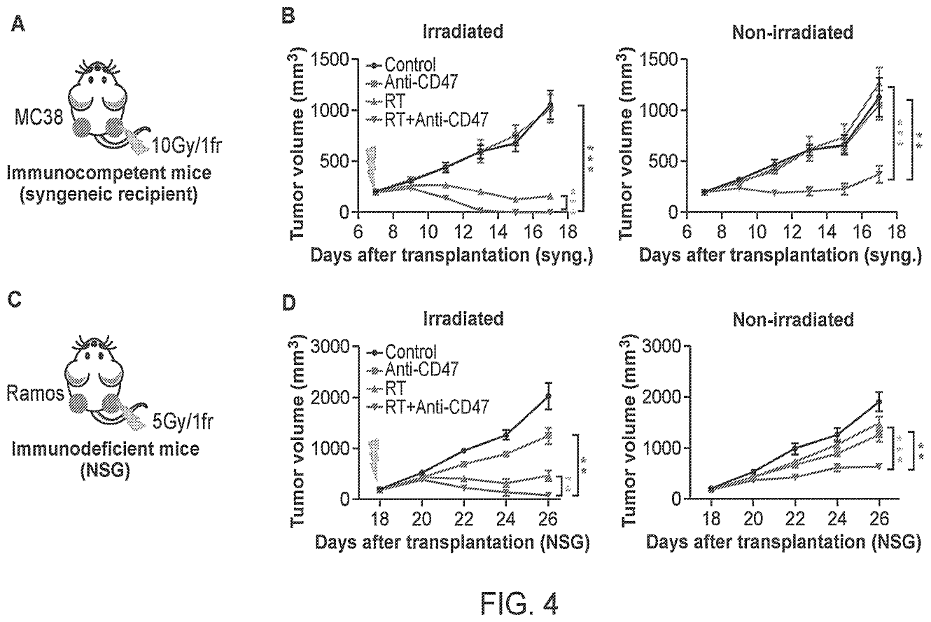

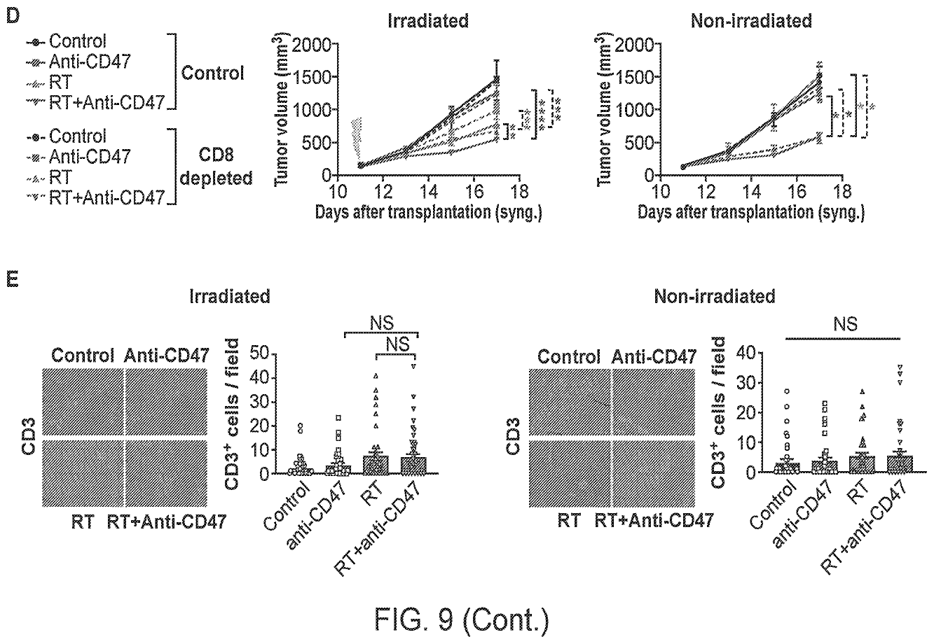

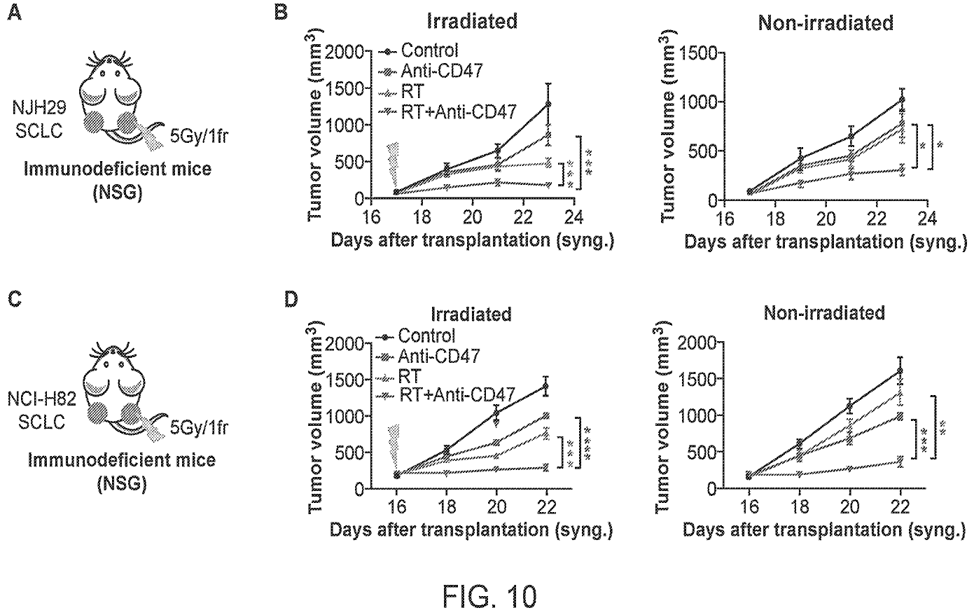

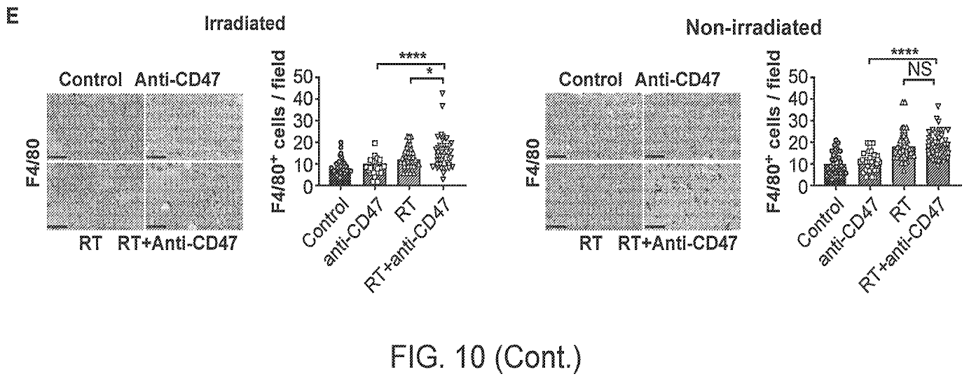

A number of studies point to a possible role for T cells, including cytotoxic CD8⁺ T cells, in abscopal effects, possibly through enhancing tumor antigen presentation and activation of. Thus, we expected that the abscopal effect observed with radiation therapy and CD47 blockade would be decreased or abolished in the absence of active cytotoxic T cells. However, when we depleted CD8⁺ T cells by in vivo treatment with anti-CD8 monoclonal antibodies (FIG. 9C and FIG. 2C), this depletion did not prevent the stimulation of an abscopal effect in mice treated with the combination therapy (FIG. 2D and FIG. 9D). Accordingly, infiltration of T cells was not increased by the combination therapy compared to either treatment alone (FIG. 9E). To further determine whether the abscopal effects observed might be independent of all T cells, we performed experiments in immunodeficient NSG mice bearing tumors from two human SCLC cell lines (NHJ29 and NCI-H82) isolated years apart from different patients. For both human models, the induction of systemic antitumor effects by radiation therapy by CD47 blockade was still observed (FIGS. 10A-D). Moreover, when mice were transplanted with each of the two cell lines, irradiation of NJH29 xenografts on one side still led to the inhibition of NCI-H82 xenografts on the contralateral unirradiated side in the combination therapy arm (FIGS. 3E-F). While endogenous T cell development may not always be completely blocked in some NSG mice, the anti-SCLC abscopal effect observed upon depletion of CD8+ T cells and in a severely immunocompromised setting using two independent cancer cell lines strongly indicates that this effect is not due to an adaptive immune system response against specific tumor antigens.

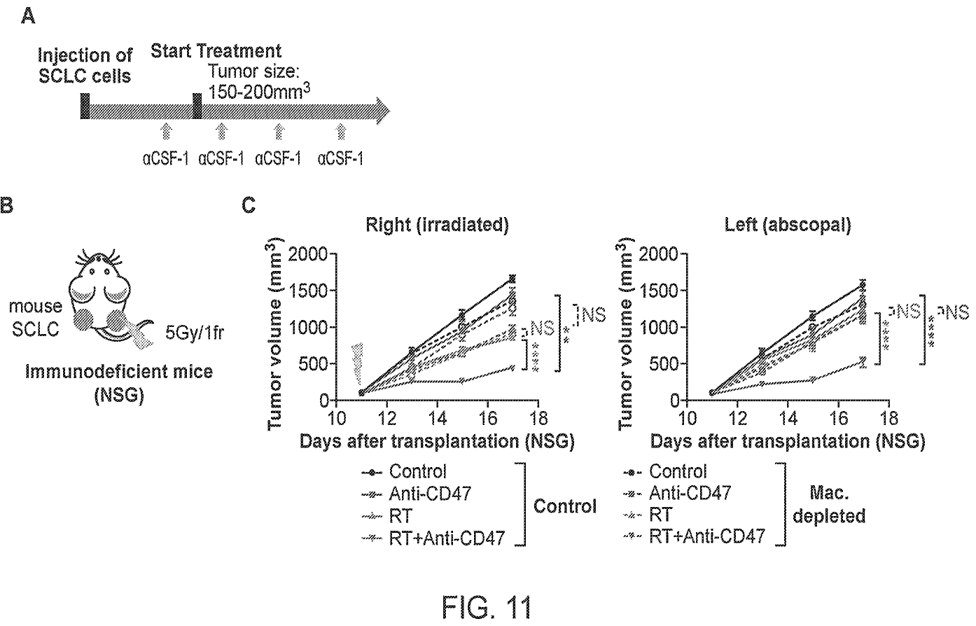

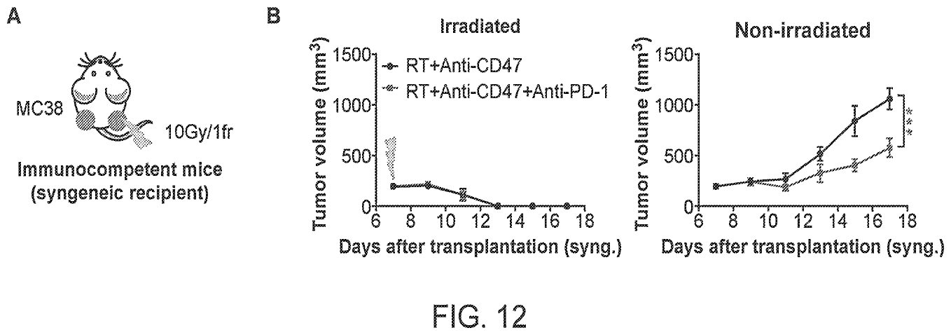

The known antitumor effects of macrophages upon CD47 blockade strongly suggested that macrophages were responsible for the antitumor abscopal effects observed in various SCLC models upon combination of radiation therapy and CD47 blockade. In support of this idea, we observed recruitment of macrophages to unirradiated tumors sites when contralateral tumors were irradiated in human xenografts (FIG. 10E). To determine the role of macrophages in these systemic effects, we depleted these cells by in vivo treatment with anti-CSF1 monoclonal antibodies in immunocompetent mice growing SCLC allografts (FIG. 11A and FIGS. 3A-B). Macrophage depletion abrogated both local and systemic effects of the combination therapy in this model (FIG. 3C). This effect correlated with the recruitment of macrophages to both irradiated and non-irradiated site upon combination of radiation therapy and CD47 blockade (FIG. 3D). To control for possible effects of CSF1 blockade on other types of immune cells, we repeated the experiment in immunodeficient NSG mice, with the same result of inhibition of the antitumor abscopal effect upon CSF-1 blockade (FIGS. 11B-C). Thus, macrophages are the key cell type mediating abscopal effects against SCLC upon irradiation.

SCLC tumors generally have low levels of infiltration with CD8⁺ T cells, which may explain the limited efficacy of treatment with anti-PD-1/PD-L1 inhibitors. In contrast, SCLC tumors can have high levels of macrophage infiltration. Here we show that combination of radiation and activation of macrophages by CD47 blockade is a promising treatment strategy for SCLC. Our results are readily tested in the clinic. In SCLC patients, the combination of radiation therapy and CD47 blockade can help treat primary tumors or metastases locally and also reduce the growth of distant lesions that are more difficult to treat with radiation therapy.

### Materials and Methods

Cell lines and culture. Human NCI-H82 SCLC cells were obtained from ATCC. NJH29 cells were described before and propagated in our laboratory. Rb/p53 mutant mouse SCLC KP1 and KP3 cells were previously described and propagated in our laboratory. KP1 Cd47 knockout and control cells were previously described. All cells were cultured in RPMI-1640 supplemented with 10% fetal bovine serum (Hyclone), 1×GlutaMax (Invitrogen), and 100 U/mL penicillin and 100 μg/mL streptomycin (Invitrogen). Cell lines were grown in suspension and dissociated by gently pipetting. Cell lines were cultured in humidified incubators at 37° C. with 5% carbon dioxide. All cell lines are routinely tested (Lonza) and confirmed to be free of mycoplasma contamination.

In vivo SCLC models. All experiments regarding use of mice were performed per protocols by the National Institute of Health at Stanford's Research animal facility. Nod.Cg-Prkdc^{scid}IL2rg^{tm1Wjl}/SzJ (NSG) mice (Jackson Laboratories, Stock No: 005557) were used for experiments in immunodeficient recipients. B6.129S F1 mice (Jackson Laboratories, Stock No: 101043) were used for were used for experiments in immunocompetent recipients. Mice were engrafted with 10⁶ cancer cells in antibiotic-free serum-free media with 1:1 mixture of Matrigel (BD Matrigel, 356237) at 6-15 weeks of age.

For allograft models, tumors were allowed to grow for 10-14 days, and then the mice were randomized into treatment groups with PBS or 150 μg anti-mouse CD47 antibody (MIAP410, Bio X Cell) every other day and/or radiation. For KP1 Cd47 knockout allografts, tumors were irradiated when the average tumor size reached around 150-300 mm³, day 10 for KP1 control and day 14 for KP1 Cd47 knockout cells in NSG mice, and day 11 for KP1 control and day 13 for KP1 Cd47 knockout cells in B6.129S F1 mice.

For xenograft models, tumors were allowed to grow for 10-14 days, and then the mice were randomized into treatment groups with PBS or 400 μg anti-human CD47 antibody (B6H12, Bio X Cell) every day and/or radiation. Mice were treated with 10 mg/kg anti-CD8α antibody (2.43, Bio X Cell) two times per week for CD8⁺ T cell depletion and 10 mg/kg anti-CSF-1 antibody (5A1, Bio X Cell) three times per week for macrophage depletion.

For all treatment models, therapeutic agents were administered by intraperitoneal injection. For all models, tumor growth was monitored by tumor dimension measurements that were used to calculate tumor volume. Tumor volumes were calculated as 0.5×length×width².

Radiation. Animal irradiation was performed using a PXi X-rad SmART cabinet irradiator (Precision X-Ray Inc., North Branford, CT). Mice were anesthetized using isoflurane through a nose cone supplied to the animal stage. Computed tomography (CT) images were acquired using a beam energy of 40 kVp, a beam filter of 2 mm AI and a voxel size of 0.2 mm. Treatment planning was performed with the RT image software package, version 3.13.1. A 10 mm collimator was used to target tumors while sparing adjacent normal tissue. Therapeutic irradiations were performed using an X-ray energy of 225 kVp and a current of 13 mA producing a dose rate of 241 cGy/min at the isocenter. The procedure described by AAPM TG-61 was used to commission and calibrate the irradiator and to ensure dosimetric accuracy through biannual quality assurance, using ion chamber and radiochromic film measurements.

RNA sequencing and analysis. For RNA-seq analysis, cell pellets were collected and sent to Novogene (https://en.novogene.com/) for RNA extraction and Illumina sequencing. Reads were quantified based on the mouse reference genome mm10 using Salmon (4) using default settings. Differentially expressed genes were obtained using DESeq2 using IHW for p-value correction. Plots were generated ggplot2. Genes were selected by filtering for log2 fold change >1.5 or <−1.5 with corrected p-value <0.05. GO pathway analysis was performed using Metascape.

Macrophage differentiation and phagocytosis assays. Mouse macrophages were differentiated as previously described. Briefly, mouse macrophages were differentiated from the bone marrow of B6.129S F1 mice. Unfractionated bone marrow cells were cultured in RPMI+GlutaMax with 10% fetal bovine serum, 100 U/mIL penicillin and 100 μg/mL streptomycin, and 10ng/mL murine M-CSF (Peprotech). In vitro phagocytosis assays were performed as previously described. Briefly, SCLC cell lines labeled with Calcein AM (Invitrogen) or FITC-conjugated beads (Cayman, 500290) were used as targets. Macrophages were washed twice with PBS, then incubated with 1×TrypLE for approximately 10 min in humidified incubator at 37° C. Macrophages were removed from the plates using gentle pipetting, then washed twice with serum-free RPMI. Phagocytosis reactions were carried out using 50,000 macrophages and 100,000 target cancer cells for 2 h in a humidified 5% CO2 incubator at 37° C. in 96-well U-bottom plates. After co-culture, cells were washed with PBS and stained with BV785-labelled anti-CD11b (CloneM1/70, BioLegend) to identify mouse macrophages. Assays were analyzed by flow cytometry using a LSRFortessa (BD Biosciences). Phagocytosis was measured as the number of CD11b⁺ CalceinAM⁺ macrophages, quantified as a percentage of total CD11b⁺ macrophages.

Flow cytometry. To create cell suspensions, tumors were removed, finely chopped, and suspended in PBS. Tumors were digested with collagenase/Dispase for 30 min at 37° C. then filtered through a 40 μm mesh. Cells were resuspended in red blood cell (RBC) lysis buffer for lmin at room temperature. Cells were resuspended in PBS, counted, Fc receptors were blocked with CD16/32 Ab (BioLegend), and then 10⁶ cells were stained with conjugated Ab cocktail for 20 min on ice. Cells were washed two times in PBS, and then resuspended for flow cytometry analysis. The following mouse Ab clones were used for analysis. CD45 (1:200, 103133, 30-F11, Biolegend), CD3 (1:200, 100327, Biolegend), CD4 (1:200, 100423, GK1.5, Biolegend), CD8 (1:200, 100708, 53-6.7, Biolegend), CD11b (1:200, 101243, M1/70, Biolegend), F4/80 (1:200, 123110, BM8, Biolegend).

Cytokine profiling. Mouse cytokine secretion was assessed in vitro. Cells were irradiated with 5Gy and cultured for 24 h, and then supernatants were collected and stored at −80° C. Mouse cytokines were analyzed by the Stanford University Human Immune Monitoring Center using a Luminex 38-plex mouse cytokine array.

Immunostaining. Tumor samples were fixed in 10% neutral buffered formalin (NBF) and embedded in paraffin before staining with hematoxylin and eosin (H&E) or immunostaining. Tumor sections were dewaxed, antigen retrieval was performed with proteinase K treatment (20 μg/ml for 15 minutes, ThermoFisher Scientific 25530049), and sections were stained with rat anti-mouse F4/80 antibody (1:50, BM8, Invitrogen). DAB was developed until precipitation was noted in specific areas of tumor sections using the HRP/DAB kit (Abcam av64238). To block non-specific signal and increase sensitivity, the Avidin/Biotin Blocking Kit (Vector laboratories) and TSA Biotin Kits (PerkinElmer) were used.

qRT-PCR. Total RNA was extracted using the RNAeasy Mini Kit (Qiagen). For RT-qPCR, 1 μg of total RNA was used to make cDNA using the NEB ProtoScript cDNA synthesis kit, and cDNA was diluted 1:20 before use. Primer information. The sequences of primers of qRP-PCR are as follows:

SEQ ID NO: 1 human CSF-1 forward:
5′-GTTTGTAGACCAGGAACAGTTGAA-3′
SEQ ID NO: 2 human CSF-1 reverse:
5′-CGCATGGTGTCCTCCATTAT-3′
SEQ ID NO: 3 human CCL2 forward
5′-CATTGTGGCCAAGGAGATCTG-3′
SEQ ID NO: 4 human CCL2 reverse
5′-CTTCGGAGTTTGGGTTTGCTT-3′
SEQ ID NO: 5 human MCP3 forward:
5′-TGTCCTTTCTCAGAGTGGTTCT-3′
SEQ ID NO: 6 human MCP3 reverse:
5′-TGCTTCCATAGGGACATCATA-3′
SEQ ID NO: 7 human GAPDH forward:
5′-CTCTCTGCTCCTCCTGTTCGAC-3′
SEQ ID NO: 8 human GAPDH reverse:
5′-TGAGCGATGTGGCTCGGCT-3′
SEQ ID NO: 9 mouse CSF-1 forward:
5′-CGGGCATCATCCTAGTCTTGCTGACTGT-3′
SEQ ID NO: 10 mouse CSF-1 reverse:
5′-ATAGTGGCAGTATGTGGGGGGCATCCTC-3′
SEQ ID NO: 11 mouse CCL2 forward
5′-GGTCCCTGTCATGCTTCTG-3′
SEQ ID NO: 12 mouse CCL2 reverse
5′-TGGTTGTGGAAAAGGTAGTGG-3′
SEQ ID NO: 13 mouse MCP3 forward
5′-CAAGAGCTACAGAAGGATCACC-3′
SEQ ID NO: 14 mouse MCP3 reverse
5′-CACTGATTCTTGCAAAGTCCC-3′
SEQ ID NO: 15 mouse GAPDH forward
5′-CAAGGTCATCCCAGAGCTGAA-3′
SEQ ID NO: 16 mouse GAPDH reverse
5′-CAGATCCACGACGGACACA-3′

Statistical analysis. Statistical significance was assayed with GraphPad Prism software. Data are represented as mean±sem. *p<0.05, **p<0.01, ***p<0.001, ****p<0.0001, ns not significant. The tests used are indicated in the figure legend. To compare growth curves, we used two-way ANOVA followed by t-tests. When comparing more than two groups, we first performed one-way ANOVA, followed by t-tests. If the F-test for variance showed a significantly different distribution between two groups being compared (F-test p<0.05), the nonparametric Mann-Whitney P value is reported instead of the Student t test P value.

### REFERENCES

1. D. A. Jaffray, Image-guided radiotherapy: from current concept to future perspectives. Nat Rev Clin Oncol 9, 688-699 (2012).
2. M. E. Lomax, L. K. Folkes, P. O'Neill, Biological consequences of radiation-induced DNA damage: relevance to radiotherapy. Clin Oncol (R Coll Radiol) 25, 578-585 (2013).
3. S. Demaria, E. B. Golden, S. C. Formenti, Role of Local Radiation Therapy in Cancer Immunotherapy. JAMA Oncol 1, 1325-1332 (2015).
4. M. McLaughlin et al., Inflammatory microenvironment remodelling by tumour cells after radiotherapy. Nat Rev Cancer 20, 203-217 (2020).
5. K. I. Jones et al., Radiation combined with macrophage depletion promotes adaptive immunity and potentiates checkpoint blockade. EMBO Mol Med 10, (2018).
6. J. Kabiljo, F. Harpain, S. Carotta, M. Bergmann, Radiotherapy as a Backbone for Novel Concepts in Cancer Immunotherapy. Cancers (Basel) 12, (2019).
7. A. B. Sharabi, M. Lim, T. L. DeWeese, C. G. Drake, Radiation and checkpoint blockade immunotherapy: radiosensitisation and potential mechanisms of synergy. Lancet Oncol 16, e498-509 (2015).
8. P. Sharma, J. P. Allison, The future of immune checkpoint therapy. Science 348, 56-61 (2015).
9. M. Vilalta, M. Rafat, A. J. Giaccia, E. E. Graves, Recruitment of circulating breast cancer cells is stimulated by radiotherapy. Cell Rep 8, 402-409 (2014).
10. J. K. Sabari, B. H. Lok, J. H. Laird, J. T. Poirier, C. M. Rudin, Unravelling the biology of SCLC: implications for therapy. Nat Rev Clin Oncol 14, 549-561 (2017).
11. J. K. Salama et al., A pooled analysis of limited-stage small-cell lung cancer patients treated with induction chemotherapy followed by concurrent platinum-based chemotherapy and 70 Gy daily radiotherapy: CALGB 30904. J Thorac Oncol 8, 1043-1049 (2013).
12. B. J. Slotman et al., Use of thoracic radiotherapy for extensive stage small-cell lung cancer: a phase 3 randomised controlled trial. Lancet 385, 36-42 (2015).
13. M. V. Mishra, A. V. Louie, V. Gondi, B. Slotman, The evolving role of radiotherapy in the management of small cell lung cancer. J Thorac Dis 10, S2545-S2554 (2018).
14. L. Horn et al., First-Line Atezolizumab plus Chemotherapy in Extensive-Stage Small-Cell Lung Cancer. N Engl J Med 379, 2220-2229 (2018).
15. P. A. Ott et al., Pembrolizumab in Patients With Extensive-Stage Small-Cell Lung Cancer: Results From the Phase Ib KEYNOTE-028 Study. J Clin Oncol 35, 3823-3829 (2017).
16. M. Feng et al., Phagocytosis checkpoints as new targets for cancer immunotherapy. Nat Rev Cancer 19, 568-586 (2019).
17. K. Weiskopf et al., CD47-blocking immunotherapies stimulate macrophage-mediated destruction of small-cell lung cancer. J Clin Invest 126, 2610-2620 (2016).
18. P. Pathria, T. L. Louis, J. A. Varner, Targeting Tumor-Associated Macrophages in Cancer. Trends Immunol 40, 310-327 (2019).
19. H. Zhou et al., Development of a micro-computed tomography-based image-guided conformal radiotherapy system for small animals. Int J Radiat Oncol Biol Phys 78, 297-305 (2010).
20. S. B. Willingham et al., The CD47-signal regulatory protein alpha (SIRPα) interaction is a therapeutic target for human solid tumors. Proc Natl Acad Sci U S A 109, 6662-6667 (2012).
21. K. Jelonek, M. Pietrowska, P. Widlak, Systemic effects of ionizing radiation at the proteome and metabolome levels in the blood of cancer patients treated with radiotherapy: the influence of inflammation and radiation toxicity. Int J Radiat Biol 93, 683-696 (2017).
22. B. P. Venkatesulu, S. Mallick, S. H. Lin, S. Krishnan, A systematic review of the influence of radiation-induced lymphopenia on survival outcomes in solid tumors. Crit Rev Oncol Hematol 123, 42-51 (2018).
23. D. P. Kingsley, An interesting case of possible abscopal effect in malignant melanoma. Br J Radiol 48, 863-866 (1975).
24. M. A. Postow et al., Immunologic correlates of the abscopal effect in a patient with melanoma. N Engl J Med 366, 925-931 (2012).
25. E. B. Golden, S. Demaria, P. B. Schiff, A. Chachoua, S. C. Formenti, An abscopal response to radiation and ipilimumab in a patient with metastatic non-small cell lung cancer. Cancer Immunol Res 1, 365-372 (2013).
26. W. Ngwa et al., Using immunotherapy to boost the abscopal effect. Nat Rev Cancer 18, 313-322 (2018).
27. Y. Liu et al., Abscopal effect of radiotherapy combined with immune checkpoint inhibitors. J Hematol Oncol 11, 104 (2018).
28. L. Deng et al., Irradiation and anti-PD-L1 treatment synergistically promote antitumor immunity in mice. J Clin Invest 124, 687-695 (2014).
29. R. Chicas-Sett, I. Morales-Orue, D. Rodriguez-Abreu, P. Lara-Jimenez, Combining radiotherapy and ipilimumab induces clinically relevant radiation-induced abscopal effects in metastatic melanoma patients: A systematic review. Clin Transl Radiat Oncol 9, 5-11 (2018).
30. M. Chen et al., Adrenergic stress constrains the development of anti-tumor immunity and abscopal responses following local radiation. Nat Commun 11, 1821 (2020).
31. D. Tseng et al., Anti-CD47 antibody-mediated phagocytosis of cancer by macrophages primes an effective antitumor T-cell response. Proc Natl Acad Sci U S A 110, 11103-11108 (2013).
32. S. Demaria et al., Ionizing radiation inhibition of distant untreated tumors (abscopal effect) is immune mediated. Int J Radiat Oncol Biol Phys 58, 862-870 (2004).
33. H. R. Rodewald, T. Brocker, C. Haller, Developmental dissociation of thymic dendritic cell and thymocyte lineages revealed in growth factor receptor mutant mice. Proc Natl Acad Sci U S A 96, 15068-15073 (1999).
34. A. Calles, G. Aguado, C. Sandoval, R. Alvarez, The role of immunotherapy in small cell lung cancer. Clin Transl Oncol 21, 961-976 (2019).
35. A. K. Eerola, Y. Soini, P. Paakko, A high number of tumor-infiltrating lymphocytes are associated with a small tumor size, low tumor stage, and a favorable prognosis in operated small cell lung carcinoma. Clin Cancer Res 6, 1875-1881 (2000).
36. B. I. Sikic et al., First-in-Human, First-in-Class Phase I Trial of the Anti-CD47 Antibody Hu5F9-G4 in Patients With Advanced Cancers. J Clin Oncol 37, 946-953 (2019).
37. R. Advani et al., CD47 Blockade by Hu5F9-G4 and Rituximab in Non-Hodgkin's Lymphoma. N Engl J Med 379, 1711-1721 (2018).
38. R. H. Mole, Whole body irradiation; radiobiology or medicine? Br J Radiol 26, 234-241 (1953).
39. N. S. Jahchan et al., A drug repositioning approach identifies tricyclic antidepressants as inhibitors of small cell lung cancer and other neuroendocrine tumors. Cancer Discov 3, 1364-1377 (2013).
40. K. S. Park et al., A crucial requirement for Hedgehog signaling in small cell lung cancer. Nat Med 17, 1504-1508 (2011).
41. K. Weiskopf et al., CD47-blocking immunotherapies stimulate macrophage-mediated destruction of small-cell lung cancer. J Clin Invest 126, 2610-2620 (2016).
42. R. Patro, G. Duggal, M. I. Love, R. A. Irizarry, C. Kingsford, Salmon provides fast and bias-aware quantification of transcript expression. Nat Methods 14, 417-419 (2017).
43. Y. Zhou et al., Metascape provides a biologist-oriented resource for the analysis of systems-level datasets. Nat Commun 10, 1523 (2019).

1. A method for the treatment of a cancer in a patient, the method comprising:
administering to said patient an effective dose of radiation therapy in combination with administration of an effective dose of a CD47 blocking agent, thereby providing for an abscopal effect causing a reduction in the number of cancer cells outside of the field of radiation.

2. The method of claim 1, wherein the cancer is a lung cancer.

3. The method of claim 1, wherein the cancer is small cell lung cancer (SCLC).

4. The method of claim 1, wherein the cancer is non-small cell lung cancer (NSCLC). (original) The method of claim 4, wherein the NSCLC is stage II, III, or IV.

6. The method of claim 1 wherein the cancer is a colorectal cancer.

7. The method of claim 1, wherein the cancer is a lymphoma.

8. The method of claim 1, wherein the cancer is metastatic.

9. The method of claim 1, wherein the CD47 blocking agent and the radiation are administered concurrently.

10. The method of claim 1, wherein the CD47 blocking agent and the radiation are administered on alternating days.

11. The method of claim 1, wherein the CD47 blocking agent is an antibody.

12. The method of claim 11, wherein the antibody specifically binds to CD47.

13. The method of claim 12, wherein the antibody is magrolimab.

14. The method of claim 11, wherein the antibody specifically binds to SIRPα.

15. The method of claim 1, wherein a series of therapeutic doses of the CD47 blocking agent are provided over the course of the radiation therapy.

16. The method of claim 1, further comprising administering an effective dose of a PD-1 blocking agent in combination with the CD47 blocking agent.

17. The method of claim 16, wherein the PD-1 blocking agent is selected from Nivolumab, pidilizumab, AMP-514, pembrolizumab, AUNP 12, anti-PD-1 monoclonal antibody clone RMP1-14 or Cemiplimab.

18. The method of claim 1, wherein the radiation therapy is delivered in a conventional fractionation scheme.

19. The method of claim 18, wherein the conventional fractionation scheme delivers up to 60 or 66 Gray (Gy) daily in fractions of from 2 to 2.75 Gy

20. The method of claim 1, wherein the radiation therapy is delivered in a fractionation scheme selected from a hyperfractionation scheme, an accelerated fractionation.
